SEQUENCE LISTING (16 sequences)

SEQ ID NO: 1 - DNA, 18 bp, Salmonella typhimurium
gaacgtgtcgcggaagtc

SEQ ID NO: 2 - DNA, 18 bp, Salmonella typhimurium
cggcaatagcgtcacctt

SEQ ID NO: 3 - DNA, 38 bp, Salmonella typhimurium
gcgcggcatccgcatcaatatctggatggtatgcccgg

SEQ ID NO: 4 - DNA, 38 bp, Salmonella typhimurium
gcgaacggcgaagcgtactgtcgcaccgtcaaaggaac

SEQ ID NO: 5 - DNA, 25 bp, Salmonella typhimurium
tcaaatcggcatcaatactcatctg

SEQ ID NO: 6 - DNA, 21 bp, Salmonella typhimurium
aaagggaaagccagctttacg

SEQ ID NO: 7 - DNA, 199 bp, Salmonella typhimurium
gaacgtgtcgcggaagtcgcggcccgattttctctggatggtatgcccggtaaacagatgagtattgatgccgatttgaaggccggtattattgatgcggatgccgcgcgcgaacggcgaagcgtactggaaagggaaagccagctttacggttcctttgacggtgcgatgaagtttatcaaaggtgacgctattgccg

SEQ ID NO: 8 - DNA, 24 bp, Salmonella typhimurium
cgtaaagggaaagccagctttacg

SEQ ID NO: 9 - DNA, 18 bp, Escherichia coli
cggcgaacagtaaggaag

SEQ ID NO: 10 - DNA, 18 bp, Escherichia coli
actggccatgactggtat

SEQ ID NO: 11 - DNA, 40 bp, Escherichia coli
tggtggttctgttagatccaacaagcatcgcggaatatgg

SEQ ID NO: 12 - DNA, 38 bp, Escherichia coli
ttcggccaacggcgatcatgaaactcctggtttatctg

SEQ ID NO: 13 - DNA, 22 bp, Escherichia coli
caaaccctacaccattatctgt

SEQ ID NO: 14 - DNA, 19 bp, Escherichia coli
gcggcgacatcattatgga

SEQ ID NO: 15 - DNA, 314 bp, Escherichia coli
cggcgaacagtaaggaaggaacaattacgttgaaatgtgataatcttttcggcgacaaaaaacaagcatcgcggaatatggttgtatatctttctagcagtgacttagttaaaggaagtaatactattttgcgtggtaaaacagataatggtgtagggtttgtgttggatctaacagaaccaccaaaagggactgaggctgccattaaaatttcggccaacggcgatcagggcgcggcgacatcattatggaaaacagataaaccaggagtttcattaaatagcaacattattaatataccagtcatggccagt

SEQ ID NO: 16 - DNA, 32 bp, Escherichia coli
tcaccatgggcgcggcgacatcattatggtga

(19) United States
(12) Patent Application Publication
(10) Pub. No.: US 2023/0366038 A1
(43) Pub. Date: Nov. 16, 2023

(54) COMPOSITIONS AND METHODS RELATING TO LOOP MEDIATED ISOTHERMAL AMPLIFICATION (LAMP)

(71) Applicant: BIOO Scientific Corporation, Austin, TX (US)

(72) Inventors: Eva May Ng, Austin, TX (US); Matthew Hooper, Austin, TX (US); Nicolas M. Kosa, Austin, TX (US); Erik Breitenfeldt, Austin, TX (US)

(21) Appl. No.: 17/745,347

(22) Filed: May 16, 2022

(51) Int. Cl. C12Q 1/689

(52) U.S. Cl. CPC C12Q 1/689; C12Q 1/6851

(57) ABSTRACT

Methods for detection of a target nucleic acid in a sample are provided according to aspects of the present disclosure which include: providing a reaction mixture comprising a LAMP assay primer set specific for the target nucleic acid, magnesium, dNTPs, a reaction buffer, a DNA polymerase, and a sample to be tested for presence of the target nucleic acid, wherein the LAMP assay primer set comprises a forward inner primer (FIP), a backward inner primer (BIP), a forward outer primer (F3) and a backward outer primer (B3), wherein the FIP and BIP are present in a non-equal ratio such that molar concentration of BIP is highly skewed relative to molar concentration of FIP. The reaction mixture is incubated under amplification reaction conditions to produce a reaction product comprising amplified target nucleic acids. The amplified target nucleic acids are then detected.

### BACKGROUND OF THE INVENTION

Current methods for detection of target nucleic acids lack sensitivity and specificity and are subject to long delays between the start of an assay and obtaining a result.

The present disclosure provides improved assays with high sensitivity and specificity, along with fast time to assay result.

### FIELD OF THE INVENTION

According to general aspects of the present disclosure, Loop Mediated Isothermal Amplification (LAMP) assays are described. According to specific aspects of the present disclosure, “lopsided” Loop Mediated Isothermal Amplification (LAMP) assays are described wherein ratios of particular primers are highly skewed relative to each other.

### SUMMARY OF THE INVENTION

Methods for detection of a target nucleic acid in a sample according to aspects of the present disclosure include: providing a reaction mixture including a LAMP assay primer set specific for the target nucleic acid, magnesium, dNTPs, a reaction buffer, a DNA polymerase, and a sample to be tested for presence of the target nucleic acid, wherein the LAMP assay primer set includes a forward inner primer (FIP), a backward inner primer (BIP), a forward outer primer (F3) and a backward outer primer (B3), wherein the FIP and BIP are present in a non-equal ratio such that molar concentration of BIP is highly skewed relative to molar concentration of FIP; incubating the reaction mixture under amplification reaction conditions to produce a reaction product including amplified target nucleic acids, wherein the reaction product includes a forward strand and a complementary backward strand, the forward strand including the FIP, the backward strand including the BIP; and detecting the amplified target nucleic acids. According to aspects of the present disclosure, BIP has a molar concentration at least 12% higher than the molar concentration of FIP and no more than 300% higher than the molar concentration of FIP, or FIP has a molar concentration at least 12% higher than the molar concentration of BIP and no more than 300% higher than the molar concentration of BIP. According to further aspects of the present disclosure, BIP has a molar concentration at least 50% higher than the molar concentration of FIP and no more than 300% higher than the molar concentration of FIP, or FIP has a molar concentration at least 50% higher than the molar concentration of BIP and no more than 300% higher than the molar concentration of BIP. According to aspects of the present disclosure, the reaction mixture further includes a reverse transcriptase.

According to aspects of the present disclosure, the F3 and B3 are present in an equal ratio. According to further aspects of the present disclosure, the F3 and B3 are present in a non-equal ratio such that when FIP is present in a greater molar concentration than BIP, F3 is present in a greater molar concentration than B3, and wherein when BIP is present in a greater molar concentration than FIP, B3 is present in a greater molar concentration than F3. According to aspects of the present disclosure, when BIP has a molar concentration at least 12% higher than the molar concentration of FIP and no more than 300% higher than the molar concentration of FIP, B3 has a molar concentration higher than the molar concentration of F3 and no more than 1000% higher than the molar concentration of F3. According to aspects of the present disclosure, when FIP has a molar concentration at least 12% higher than the molar concentration of BIP and no more than 300% higher than the molar concentration of BIP, F3 has a molar concentration higher than the molar concentration of B3 and no more than 1000% higher than the molar concentration of B3.

According to further aspects of the present disclosure, when BIP has a molar concentration at least 50% higher than the molar concentration of FIP and no more than 300% higher than the molar concentration of FIP, B3 has a molar concentration higher than the molar concentration of F3 and no more than 1000% higher than the molar concentration of F3. According to further aspects of the present disclosure, when FIP has a molar concentration at least 50% higher than the molar concentration of BIP and no more than 300% higher than the molar concentration of BIP, F3 has a molar concentration higher than the molar concentration of B3 and no more than 1000% higher than the molar concentration of B3.

According to aspects of the present disclosure, the reaction mixture further includes a Loop F (LF) primer, a Loop B (LB) primer, or both a Loop F (LF) primer and a Loop B (LB) primer, wherein the LF and LB are present in a non-equal ratio when both are present, such that molar concentration of LF is highly skewed relative to LB. According to aspects of the present disclosure, when BIP has a molar concentration at least 12% higher than the molar concentration of FIP and no more than 300% higher than the molar concentration of FIP, LB is present and no LF is present, or LB has a molar concentration at least 12% higher than the molar concentration of LF and no more than 500% higher than the molar concentration of LF when both LB and LF are present. According to aspects of the present disclosure, when FIP has a molar concentration at least 12% higher than the molar concentration of BIP and no more than 300% higher than the molar concentration of BIP, LF is present and no LB is present, or LF has a molar concentration at least 12% higher than the molar concentration of LB and no more than 500% higher than the molar concentration of LB when both LB and LF are present.

According to further aspects of the present disclosure, when BIP has a molar concentration at least 50% higher than the molar concentration of FIP and no more than 300% higher than the molar concentration of FIP, LB is present and no LF is present, or LB has a molar concentration at least 50% higher than the molar concentration of LF and no more than 500% higher than the molar concentration of LF when both LB and LF are present. According to further aspects of the present disclosure, when FIP has a molar concentration at least 50% higher than the molar concentration of BIP and no more than 300% higher than the molar concentration of BIP, LF is present and no LB is present, or LF has a molar concentration at least 50% higher than the molar concentration of LB and no more than 500% higher than the molar concentration of LB when both LB and LF are present.

According to aspects of the present disclosure, detecting the amplified target nucleic acids includes detecting one or more of: pH, turbidity, an electrophoresis pattern, and a detectable label.

According to aspects of the present disclosure, detecting the amplified target nucleic acids includes detecting a detectable label, wherein the detectable label includes one or more of: a fluorescent label, a bioluminescent label, a chemiluminescent label, a chromophore, a magnetic label, an enzyme, a substrate, and a radioisotope.

According to aspects of the present disclosure, the detectable label includes a fluorescence resonance energy transfer (FRET) fluorescent label.

According to aspects of the present disclosure, the detectable label includes a fluorescent intercalating dye.

According to aspects of the present disclosure, the detectable label is present in one or more primers of the LAMP assay primer set.

According to aspects of the present disclosure, the detectable label is a fluorescent label present in one or more primers of the LAMP assay primer set, incorporated into the amplified target nucleic acids.

According to aspects of the present disclosure, the detectable label is a fluorescent label present in a probe, wherein the probe is a sequence-specific binding partner for a specified sequence present in the amplified target nucleic acids.

According to aspects of the present disclosure, the probe is a sequence-specific binding partner for a specified sequence present in the forward strand of the amplified target nucleic acids when FIP has a molar concentration at least 12% higher than the molar concentration of BIP and no more than 300% higher than the molar concentration of BIP.

According to aspects of the present disclosure, the probe is a sequence-specific binding partner for a specified sequence present in the forward strand of the amplified target nucleic acids when FIP has a molar concentration at least 50% higher than the molar concentration of BIP and no more than 300% higher than the molar concentration of BIP.

According to aspects of the present disclosure, the probe is a sequence-specific binding partner for a specified sequence present in the backward strand of the amplified target nucleic acids when BIP has a molar concentration at least 12% higher than the molar concentration of FIP and no more than 300% higher than the molar concentration of FIP.

According to aspects of the present disclosure, the probe is a sequence-specific binding partner for a specified sequence present in the backward strand of the amplified target nucleic acids when BIP has a molar concentration at least 50% higher than the molar concentration of FIP and no more than 300% higher than the molar concentration of FIP.

According to aspects of the present disclosure, the probe is a fluorescence resonance energy transfer (FRET)-labeled oligonucleotide probe, wherein the FRET label includes a quencher and a fluorescent moiety such that the quencher quenches a fluorescent signal of the fluorescent moiety when the oligonucleotide probe is in stem-loop configuration and not hybridized to the specified sequence, and wherein the fluorescent signal of the fluorescent moiety is detectable when the fluorescently-labeled oligonucleotide probe is specifically hybridized to the specified sequence.

Methods for detection of a target nucleic acid in a sample according to aspects of the present disclosure include: providing a reaction mixture including: a LAMP assay primer set specific for the target nucleic acid, magnesium, dNTPs, a reaction buffer, a DNA polymerase, and a sample to be tested for presence of the target nucleic acid, wherein the LAMP assay primer set includes 1) a forward inner primer (FIP), 2) a backward inner primer (BIP), 3) a forward outer primer (F3), 4) a backward outer primer (B3), and 5) a Loop F (LF) primer or a Loop B (LB) primer, or both a Loop F (LF) primer and a Loop B (LB) primer, wherein the FIP and BIP are present in a non-equal ratio such that molar concentration of BIP is highly skewed relative to molar concentration of FIP, wherein when FIP is present in a greater molar concentration than BIP, LF is present in a greater molar concentration than LB, and wherein when BIP is present in a greater molar concentration than FIP, LB is present in a greater molar concentration than LF; incubating the reaction mixture under amplification reaction conditions to produce a reaction product including amplified target nucleic acids, wherein the reaction product includes a forward strand and a complementary backward strand, the forward strand including the FIP, the backward strand including the BIP; and detecting the amplified target nucleic acids by detection of specific binding of a fluorescence resonance energy transfer (FRET)-labeled oligonucleotide probe, wherein the oligonucleotide probe is a sequence-specific binding partner for a sequence present in the forward strand or the backward strand of the amplified target nucleic acids, wherein the oligonucleotide probe is a sequence-specific binding partner for a specified sequence present in the forward strand of the amplified target nucleic acids when FIP has a molar concentration at least 12% higher than the molar concentration of BIP, wherein the oligonucleotide probe is a sequence-specific binding partner for a specified sequence present in the backward strand of the amplified target nucleic acids when BIP has a molar concentration at least 12% higher than the molar concentration of FIP, wherein the FRET label includes a quencher and a fluorescent moiety such that the quencher quenches a fluorescent signal of the fluorescent moiety when the oligonucleotide probe is in stem-loop configuration and not hybridized to the specified sequence, and wherein the fluorescent signal of the fluorescent moiety is detectable when the fluorescently-labeled oligonucleotide probe is specifically hybridized to the specified sequence. According to aspects of the present disclosure, the reaction mixture further includes a reverse transcriptase.

According to aspects of the present disclosure, BIP has a molar concentration at least 12% higher than the molar concentration of FIP and no more than 300% higher than the molar concentration of FIP, or FIP has a molar concentration at least 12% higher than the molar concentration of BIP and no more than 300% higher than the molar concentration of BIP.

According to aspects of the present disclosure, BIP has a molar concentration at least 50% higher than the molar concentration of FIP and no more than 300% higher than the molar concentration of FIP, or FIP has a molar concentration at least 50% higher than the molar concentration of BIP and no more than 300% higher than the molar concentration of BIP.

According to aspects of the present disclosure, the F3 and B3 are present in an equal ratio. According to aspects of the present disclosure, the F3 and B3 are present in a non-equal ratio such that when FIP is present in a greater molar concentration than BIP, F3 is present in a greater molar concentration than B3, and wherein when BIP is present in a greater molar concentration than FIP, B3 is present in a greater molar concentration than F3.

According to aspects of the present disclosure, when BIP has a molar concentration at least 12% higher than the molar concentration of FIP and no more than 300% higher than the molar concentration of FIP, B3 has a molar concentration higher than the molar concentration of F3 and no more than 1000% higher than the molar concentration of F3. According to aspects of the present disclosure, when FIP has a molar concentration at least 12% higher than the molar concentration of BIP and no more than 300% higher than the molar concentration of BIP, F3 has a molar concentration higher than the molar concentration of B3 and no more than 1000% higher than the molar concentration of B3.

According to further aspects of the present disclosure, when BIP has a molar concentration at least 50% higher than the molar concentration of FIP and no more than 300% higher than the molar concentration of FIP, B3 has a molar concentration higher than the molar concentration of F3 and no more than 1000% higher than the molar concentration of F3. According to further aspects of the present disclosure, when FIP has a molar concentration at least 50% higher than the molar concentration of BIP and no more than 300% higher than the molar concentration of BIP, F3 has a molar concentration higher than the molar concentration of B3 and no more than 1000% higher than the molar concentration of B3.

According to aspects of the present disclosure, when BIP has a molar concentration at least 12% higher than the molar concentration of FIP and no more than 300% higher than the molar concentration of FIP, LB is present and no LF is present, or LB has a molar concentration at least 12% higher than the molar concentration of LF and no more than 500% higher than the molar concentration of LF when both LB and LF are present. According to aspects of the present disclosure, when FIP has a molar concentration at least 12% higher than the molar concentration of BIP and no more than 300% higher than the molar concentration of BIP, LF is present and no LB is present, or LF has a molar concentration at least 12% higher than the molar concentration of LB and no more than 500% higher than the molar concentration of LB when both LB and LF are present.

According to further aspects of the present disclosure, when BIP has a molar concentration at least 50% higher than the molar concentration of FIP and no more than 300% higher than the molar concentration of FIP, LB is present and no LF is present, or LB has a molar concentration at least 50% higher than the molar concentration of LF and no more than 500% higher than the molar concentration of LF when both LB and LF are present. According to further aspects of the present disclosure, when FIP has a molar concentration at least 50% higher than the molar concentration of BIP and no more than 300% higher than the molar concentration of BIP, LF is present and no LB is present, or LF has a molar concentration at least 50% higher than the molar concentration of LB and no more than 500% higher than the molar concentration of LB when both LB and LF are present.

### BRIEF DESCRIPTION OF THE DRAWINGS

FIG. 1 is a graph which shows results of LAMP assays in which samples #1-8 were the results using a master mix containing the concentrations as stated in Primer Mix A in Table 1 and which shows results of LAMP assays in which samples #9-16 were the results of a master mix containing the concentrations as stated in Primer Mix B, Table 2;

FIG. 2A is a graph showing the real-time amplification curve output from a dilution series of target genomic DNA at concentrations ranging from 10\hat{ }6 to 10\hat{ }1, samples 1, 2, 3, 4, 5, and 6, respectively;

FIG. 2B is a graph showing the real-time amplification curve output from a dilution series of target genomic DNA at concentrations ranging from 10\hat{ }6 to 10\hat{ }1, samples 9, 10, 11, 12, 13, and 14, respectively;

FIG. 3 is a graph showing results of LAMP assays in which samples #2 and #4 were obtained using a master mix containing the concentrations stated in Primer Mix A in Table 1 and showing results of LAMP assays in which samples #6 and #8 were obtained using a master mix containing the concentrations stated in Primer Mix B, Table 2;

FIG. 4A is a graph showing the amplification curves of the corresponding data for sample 2 shown in the graph of FIG. 3;

FIG. 4B is a graph showing the amplification curves of the corresponding data for sample 4 shown in the graph of FIG. 3;

FIG. 4C is a graph showing the amplification curves of the corresponding data for sample 6 shown in the graph of FIG. 3;

FIG. 4D is a graph showing the amplification curves of the corresponding data for sample 8 shown in the graph of FIG. 3;

FIG. 5 shows amplification curves demonstrating transferable utility from one LAMP primer set to another;

FIG. 6 is a graph showing TTH defined as the time elapsed until the amplification curve reached a minimum threshold height (5000 RFU), as shown in FIG. 5;

FIG. 7 is a graph showing direct comparison of LAMP assay performance differences in sensitivity;

FIG. 8 is a graph showing results of LAMP assays of the present disclosure in which the concentration of molecular beacon (MB-B) was varied from 0.1 micromolar to 0.5 micromolar;

FIG. 9 is a graph showing results of LAMP assays using Primer Mix A, Table 1, except for variation in FIP and BIP ratios, FIP:BIP, sample IDs 1-32, which were varied as shown in Table 6;

FIG. 10 is a graph showing results of LAMP assays using Primer Mix A, Table 1, except for variation in FIP and BIP ratios, FIP:BIP, sample IDs 1-32, which were varied as shown in Table 7;

FIG. 11 is a graph showing results of LAMP assays using Primer Mix B, Table 2, except for variation in LF and LB ratios, LF:LB, sample IDs 1-32, which were varied as shown in Table 8; and

FIG. 12 is a graph showing results of LAMP assays using Primer Mix B, Table 2, except for variation in F3 and B3 ratios, F3:B3, sample IDs 1-32, which were varied as shown in Table 9.

### DETAILED DESCRIPTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; CRISPR/Cas: A Laboratory Manual, Doudna and Mali (eds), Cold Spring Harbor Laboratory Press, Cold Spring Harbor, NY, USA, 2016; D. L. Nelson and M. M. Cox, Lehninger Principles of Biochemistry, 4th Ed., W. H. Freeman & Company, 2004; Herdewijn, P. (Ed.), Oligonucleotide Synthesis: Methods and Applications, Methods in Molecular Biology, Humana Press, 2004; Remington: The Science and Practice of Pharmacy, Lippincott Williams & Wilkins, 21st Ed., 2005; L. V. Allen, Jr. et al., Ansel's Pharmaceutical Dosage Forms and Drug Delivery Systems, 8th Ed., Philadelphia, PA: Lippincott, Williams & Wilkins, 2004; and L. Brunton et al., Goodman & Gilman's The Pharmacological Basis of Therapeutics, McGraw-Hill Professional, 12th Ed., 2011.

The singular terms “a,” “an,” and “the” are not intended to be limiting and include plural referents unless explicitly stated otherwise or the context clearly indicates otherwise.

The terms “includes,” “comprises,” “including,” “comprising,” “has,” “having,” and grammatical variations thereof, when used in this specification, are not intended to be limiting, and specify the presence of stated features, elements, and/or components, but do not preclude the presence or addition of one or more other features, elements, components, and/or groups thereof.

The term “about” as used herein in reference to a number is used herein to include numbers which are greater, or less than, a stated or implied value by 1%, 5%, 10%, or 20%.

Particular combinations of features are recited in the claims and/or disclosed in the specification, and these combinations of features are not intended to limit the disclosure of various aspects. Combinations of such features not specifically recited in the claims and/or disclosed in the specification. Although each dependent claim listed below may directly depend on only one claim, the disclosure of various aspects includes each dependent claim in combination with every other claim in the claim set. As used herein, a phrase referring to “at least one of” a list of items refers to any combination of those items, including single members. As an example, “at least one of: a, b, or c” is intended to cover a alone; b alone; c alone, a and b, a, b, and c, b and c, a and c, as well as any combination with multiples of the same element, such as a and a; a, a, and a; a, a, and b; a, a, and c; a, b, and b; a, c, and c; and any other combination or ordering of a, b, and c).

Methods for detection of a target nucleic acid in a sample are provided according to aspects of the present disclosure which include loop mediated isothermal amplification (LAMP) of the target nucleic acid.

The terms “amplify, “amplification,” and “amplifying” are used to refer generally to a method or technique for copying a template nucleic acid, thereby producing nucleic acids including copies of all or a portion of the target nucleic acid.

LAMP reactions use a DNA polymerase with strand displacement activity, and typically use either 4 primers or six primers. According to aspects of the present disclosure, LAMP reactions are described which use 4 primers, 6 primers, or in some cases, 5 primers, the primers together termed a “LAMP assay primer set” herein.

The LAMP assay primer set includes at least: 1) a forward inner primer (FIP), 2) a backward inner primer (BIP), 3) a forward outer primer (F3), and 4) a backward outer primer (B3), wherein the FIP and BIP are present in a non-equal ratio such that molar concentration of BIP is highly skewed relative to molar concentration of FIP.

According to aspects of the present disclosure, an included LAMP assay primer set further includes 5) a Loop F (LF) primer, 6) a Loop B (LB) primer, or 7) both a Loop F (LF) primer and a Loop B (LB) primer. The LF and LB are present in a non-equal ratio when both are present, such that molar concentration of LF is highly skewed relative to LB.

LAMP reactions may be used according to aspects of the present disclosure to detect a DNA target or an RNA target (RT-LAMP).

Methods for detection of a target nucleic acid in a sample are provided according to aspects of the present disclosure which include providing a reaction mixture including a LAMP assay primer set specific for the target nucleic acid. The LAMP reaction mixture is incubated under LAMP isothermal amplification reaction conditions to produce a LAMP reaction product comprising amplified target nucleic acids, wherein the reaction product comprises a forward strand and a complementary backward strand, the forward strand comprising the FIP, the backward strand comprising the BIP. The LAMP amplified target nucleic acids, i.e. the LAMP reaction products, are then detected.

The term “primer” as used herein refers to an oligonucleotide capable of acting as a point of initiation of enzymatic synthesis of an oligonucleotide primer extension product under conditions in which synthesis of an oligonucleotide primer extension product which is complementary to a target nucleic acid is induced. Such conditions include the presence of nucleotides, with or without one or more nucleotide analogs, and presence of a suitable polymerase and any needed polymerase cofactors such as magnesium ions, in a reaction mixture at a suitable temperature and pH. Primers are designed according to well-known methods and criteria. For instance, the annealing temperature of the primers should be about the same, within a few degrees, and the primers should not form dimers with each other. Specific primers may be designed by analysis of nucleic acid sequences of a target organism such as by use of CLUSTAL X or a similar program. Specificity of designed primers may be confirmed by comparison with a nucleic acid sequence database, such as GenBank. Primers may be synthesized using well-known methods.

Methods and considerations for primer design are described in detail in Yuryev, A., PCR Primer Design, Methods in Molecular Biology, vol. 42, Human Press, 2007; C. W. Dieffenbach et al., PCR Primer: A Laboratory Manual, Cold Spring Harbor Laboratory Press, 2003; and V. Demidov et al., DNA Amplification: Current Technologies and Applications, Taylor & Francis, 2004.

The term “highly skewed” as used herein to describe a non-equal ratio of two LAMP primers present in a LAMP reaction mixture wherein one of the two primers is present in an amount that differs by comparison with the other of the two primers by at least about 12%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 400%, at least about 500%, at least about 600%, or more.

According to aspects of the present disclosure, BIP has a molar concentration at least about 12% higher than the molar concentration of FIP and no more than about 300% higher than the molar concentration of FIP, or FIP has a molar concentration at least about 12% higher than the molar concentration of BIP and no more than about 300% higher than the molar concentration of BIP.

According to aspects of the present disclosure, BIP has a molar concentration at least about 50% higher than the molar concentration of FIP and no more than about 300% higher than the molar concentration of FIP, or FIP has a molar concentration at least about 50% higher than the molar concentration of BIP and no more than about 300% higher than the molar concentration of BIP.

According to aspects of the present disclosure, BIP and FIP both have a concentration in the range of about 0.4 micromolar to 2.5 micromolar, wherein BIP has a molar concentration at least about 12% higher than the molar concentration of FIP and no more than about 300% higher than the molar concentration of FIP, or FIP has a molar concentration at least about 12% higher than the molar concentration of BIP and no more than about 300% higher than the molar concentration of BIP.

According to aspects of the present disclosure, BIP and FIP both have a concentration in the range of about 0.4 micromolar to 2.5 micromolar, wherein BIP has a molar concentration at least about 50% higher than the molar concentration of FIP and no more than about 300% higher than the molar concentration of FIP, or FIP has a molar concentration at least about 50% higher than the molar concentration of BIP and no more than about 300% higher than the molar concentration of BIP.

According to aspects of the present disclosure, BIP has a molar concentration at least about 12% higher than the molar concentration of FIP and no more than about 90% higher than the molar concentration of FIP, or FIP has a molar concentration at least about 12% higher than the molar concentration of BIP and no more than about 90% higher than the molar concentration of BIP.

According to aspects of the present disclosure, BIP has a molar concentration at least about 50% higher than the molar concentration of FIP and no more than about 90% higher than the molar concentration of FIP, or FIP has a molar concentration at least about 50% higher than the molar concentration of BIP and no more than about 90% higher than the molar concentration of BIP.

According to aspects of the present disclosure, BIP and FIP both have a concentration in the range of about 0.4 micromolar to 2.5 micromolar, wherein BIP has a molar concentration at least about 12% higher than the molar concentration of FIP and no more than about 90% higher than the molar concentration of FIP, or FIP has a molar concentration at least about 12% higher than the molar concentration of BIP and no more than about 90% higher than the molar concentration of BIP.

According to aspects of the present disclosure, BIP and FIP both have a concentration in the range of about 0.4 micromolar to 2.5 micromolar, wherein BIP has a molar concentration at least about 50% higher than the molar concentration of FIP and no more than about 90% higher than the molar concentration of FIP, or FIP has a molar concentration at least about 50% higher than the molar concentration of BIP and no more than about 90% higher than the molar concentration of BIP.

According to aspects of the present disclosure, F3 and B3 are present in an equal ratio or F3 and B3 are present in a non-equal ratio such that when FIP is present in a greater molar concentration than BIP, F3 is present in a greater molar concentration than B3, and wherein when BIP is present in a greater molar concentration than FIP, B3 is present in a greater molar concentration than F3.

According to aspects of the present disclosure, when BIP has a molar concentration at least about 12% higher than the molar concentration of FIP and no more than about 300% higher than the molar concentration of FIP, B3 has a molar concentration higher than the molar concentration of F3 and no more than about 1000% higher than the molar concentration of F3.

According to aspects of the present disclosure, when BIP has a molar concentration at least about 50% higher than the molar concentration of FIP and no more than about 300% higher than the molar concentration of FIP, B3 has a molar concentration higher than the molar concentration of F3 and no more than about 1000% higher than the molar concentration of F3.

According to aspects of the present disclosure, when BIP has a molar concentration at least about 12% higher than the molar concentration of FIP and no more than about 90% higher than the molar concentration of FIP, B3 has a molar concentration higher than the molar concentration of F3 and no more than about 1000% higher than the molar concentration of F3.

According to aspects of the present disclosure, when BIP has a molar concentration at least about 50% higher than the molar concentration of FIP and no more than about 90% higher than the molar concentration of FIP, B3 has a molar concentration higher than the molar concentration of F3 and no more than about 1000% higher than the molar concentration of F3.

According to aspects of the present disclosure, when FIP has a molar concentration at least about 12% higher than the molar concentration of BIP and no more than about 300% higher than the molar concentration of BIP, F3 has a molar concentration higher than the molar concentration of B3 and no more than about 1000% higher than the molar concentration of B3.

According to aspects of the present disclosure, when FIP has a molar concentration at least about 50% higher than the molar concentration of BIP and no more than about 300% higher than the molar concentration of BIP, F3 has a molar concentration higher than the molar concentration of B3 and no more than about 1000% higher than the molar concentration of B3.

According to aspects of the present disclosure, when FIP has a molar concentration at least about 12% higher than the molar concentration of BIP and no more than about 90% higher than the molar concentration of BIP, F3 has a molar concentration higher than the molar concentration of B3 and no more than about 1000% higher than the molar concentration of B3.

According to aspects of the present disclosure, when FIP has a molar concentration at least about 50% higher than the molar concentration of BIP and no more than about 90% higher than the molar concentration of BIP, F3 has a molar concentration higher than the molar concentration of B3 and no more than about 1000% higher than the molar concentration of B3.

According to aspects of the present disclosure, the reaction mixture further comprises a Loop F (LF) primer, a Loop B (LB) primer, or both a Loop F (LF) primer and a Loop B (LB) primer, wherein the LF and LB are present in a non-equal ratio when both are present, such that molar concentration of LF is highly skewed relative to LB.

According to aspects of the present disclosure, BIP has a molar concentration at least about 12% higher than the molar concentration of FIP and no more than about 300% higher than the molar concentration of FIP, LB is present and no LF is present, or LB has a molar concentration at least about 110% higher than the molar concentration of LF and no more than about 600% higher than the molar concentration of LF when both LB and LF are present.

According to aspects of the present disclosure, BIP has a molar concentration at least about 50% higher than the molar concentration of FIP and no more than about 300% higher than the molar concentration of FIP, LB is present and no LF is present, or LB has a molar concentration at least about 110% higher than the molar concentration of LF and no more than about 600% higher than the molar concentration of LF when both LB and LF are present.

According to aspects of the present disclosure, BIP and FIP both have a concentration in the range of about 0.4 micromolar to about 2.5 micromolar, BIP has a molar concentration at least about 12% higher than the molar concentration of FIP and no more than about 300% higher than the molar concentration of FIP, LB is present and no LF is present, wherein LB has a concentration in the range of about 0.4 micromolar to about 2.5 micromolar; or LB has a molar concentration at least about 110% higher than the molar concentration of LF and no more than about 600% higher than the molar concentration of LF when both LB and LF are present, wherein LB and LF both have a concentration in the range of about 0.4 micromolar to about 2.5 micromolar.

According to aspects of the present disclosure, BIP and FIP both have a concentration in the range of about 0.4 micromolar to about 2.5 micromolar, BIP has a molar concentration at least about 50% higher than the molar concentration of FIP and no more than about 300% higher than the molar concentration of FIP, LB is present and no LF is present, wherein LB has a concentration in the range of about 0.4 micromolar to about 2.5 micromolar; or LB has a molar concentration at least about 110% higher than the molar concentration of LF and no more than about 600% higher than the molar concentration of LF when both LB and LF are present, wherein LB and LF both have a concentration in the range of about 0.4 micromolar to about 2.5 micromolar.

According to aspects of the present disclosure, BIP has a molar concentration at least about 12% higher than the molar concentration of FIP and no more than about 90% higher than the molar concentration of FIP, LB is present and no LF is present, or LB has a molar concentration at least about 110% higher than the molar concentration of LF and no more than about 600% higher than the molar concentration of LF when both LB and LF are present.

According to aspects of the present disclosure, BIP has a molar concentration at least about 50% higher than the molar concentration of FIP and no more than about 90% higher than the molar concentration of FIP, LB is present and no LF is present, or LB has a molar concentration at least about 110% higher than the molar concentration of LF and no more than about 600% higher than the molar concentration of LF when both LB and LF are present.

According to aspects of the present disclosure, BIP and FIP both have a concentration in the range of about 0.4 micromolar to about 2.5 micromolar, BIP has a molar concentration at least about 12% higher than the molar concentration of FIP and no more than about 90% higher than the molar concentration of FIP, LB is present and no LF is present, wherein LB has a concentration in the range of about 0.4 micromolar to about 2.5 micromolar; or LB has a molar concentration at least about 110% higher than the molar concentration of LF and no more than about 600% higher than the molar concentration of LF when both LB and LF are present, wherein LB and LF both have a concentration in the range of about 0.4 micromolar to about 2.5 micromolar.

According to aspects of the present disclosure, BIP and FIP both have a concentration in the range of about 0.4 micromolar to about 2.5 micromolar, BIP has a molar concentration at least about 50% higher than the molar concentration of FIP and no more than about 90% higher than the molar concentration of FIP, LB is present and no LF is present, wherein LB has a concentration in the range of about 0.4 micromolar to about 2.5 micromolar; or LB has a molar concentration at least about 110% higher than the molar concentration of LF and no more than about 600% higher than the molar concentration of LF when both LB and LF are present, wherein LB and LF both have a concentration in the range of about 0.4 micromolar to about 2.5 micromolar.

According to aspects of the present disclosure, when FIP has a molar concentration at least about 12% higher than the molar concentration of BIP and no more than about 300% higher than the molar concentration of BIP, LF is present and no LB is present, or LF has a molar concentration at least about 110% higher than the molar concentration of LB and no more than about 600% higher than the molar concentration of LB when both LB and LF are present.

According to aspects of the present disclosure, when FIP has a molar concentration at least about 50% higher than the molar concentration of BIP and no more than about 300% higher than the molar concentration of BIP, LF is present and no LB is present, or LF has a molar concentration at least about 110% higher than the molar concentration of LB and no more than about 600% higher than the molar concentration of LB when both LB and LF are present.

According to aspects of the present disclosure, BIP and FIP both have a concentration in the range of about 0.4 micromolar to about 2.5 micromolar, when FIP has a molar concentration at least about 12% higher than the molar concentration of BIP and no more than about 300% higher than the molar concentration of BIP, LF is present and no LB is present; or LF has a molar concentration at least about 110% higher than the molar concentration of LB and no more than about 600% higher than the molar concentration of LB when both LB and LF are present, wherein LB and LF both have a concentration in the range of about 0.4 micromolar to about 2.5 micromolar.

According to aspects of the present disclosure, BIP and FIP both have a concentration in the range of about 0.4 micromolar to about 2.5 micromolar, when FIP has a molar concentration at least about 50% higher than the molar concentration of BIP and no more than about 300% higher than the molar concentration of BIP, LF is present and no LB is present; or LF has a molar concentration at least about 110% higher than the molar concentration of LB and no more than about 600% higher than the molar concentration of LB when both LB and LF are present, wherein LB and LF both have a concentration in the range of about 0.4 micromolar to about 2.5 micromolar.

According to aspects of the present disclosure, when FIP has a molar concentration at least about 12% higher than the molar concentration of BIP and no more than about 90% higher than the molar concentration of BIP, LF is present and no LB is present, or LF has a molar concentration at least about 110% higher than the molar concentration of LB and no more than about 600% higher than the molar concentration of LB when both LB and LF are present.

According to aspects of the present disclosure, when FIP has a molar concentration at least about 50% higher than the molar concentration of BIP and no more than about 90% higher than the molar concentration of BIP, LF is present and no LB is present, or LF has a molar concentration at least about 110% higher than the molar concentration of LB and no more than about 600% higher than the molar concentration of LB when both LB and LF are present.

According to aspects of the present disclosure, BIP and FIP both have a concentration in the range of about 0.4 micromolar to about 2.5 micromolar, when FIP has a molar concentration at least about 12% higher than the molar concentration of BIP and no more than about 90% higher than the molar concentration of BIP, LF is present and no LB is present; or LF has a molar concentration at least about 110% higher than the molar concentration of LB and no more than about 600% higher than the molar concentration of LB when both LB and LF are present, wherein LB and LF both have a concentration in the range of about 0.4 micromolar to about 2.5 micromolar.

According to aspects of the present disclosure, BIP and FIP both have a concentration in the range of about 0.4 micromolar to about 2.5 micromolar, when FIP has a molar concentration at least about 50% higher than the molar concentration of BIP and no more than about 90% higher than the molar concentration of BIP, LF is present and no LB is present; or LF has a molar concentration at least about 110% higher than the molar concentration of LB and no more than about 600% higher than the molar concentration of LB when both LB and LF are present, wherein LB and LF both have a concentration in the range of about 0.4 micromolar to about 2.5 micromolar.

According to aspects of the present disclosure, a molecular beacon probe in included in a reaction mixture, wherein the molecular beacon probe is present in a concentration in the range of about 0.1 micromolar to 0.3 micromolar.

According to aspects of the present disclosure, a molecular beacon probe in included in a reaction mixture, wherein the molecular beacon probe is present in a ratio of LF primer:molecular beacon—backwards probe in the range of about 4:1 to about 25:1.

According to aspects of the present disclosure, a molecular beacon probe in included in a reaction mixture, wherein the molecular beacon probe is present in a ratio of LB primer:molecular beacon—forward probe in the range of about 4:1 to about 25:1.

A reaction mixture according to aspects of the present disclosure includes magnesium ions, dNTPs, a reaction buffer, a DNA polymerase, and a sample to be tested for presence of the target nucleic acid.

Magnesium ions included in a reaction mixture according to aspects of the present disclosure may be included as a magnesium salt, including, but not limited to, magnesium sulfate, magnesium chloride, magnesium acetate, or any of two or more thereof. A magnesium salt is included in a reaction mixture in an amount in the range of about 0.05 mM to about 100 mM according to aspects of the present disclosure. A magnesium salt is included in a reaction buffer in an amount in the range of about 0.1 mM to about 75 mM, such as about 1 mM to about 50 mM, such as about 5 mM to about 20 mM, such as about 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, or more, or less, according to aspects of the present disclosure.

According to aspects of the present disclosure, the reaction mixture includes nucleotides and/or analogs thereof, wherein the nucleotides and analogs are suitable for incorporation by a polymerase into a nucleic acid sequence. The terms “nucleic acid sequence” and “nucleotide sequence” are used to refer to the ordering of nucleotides in an oligonucleotide or polynucleotide.

Nucleotides included in a reaction mixture according to aspects of the present disclosure include deoxyribonucleotide triphosphate molecules (dNTPs), and/or one or more analogs thereof. The term “dNTP” is an abbreviation for “a deoxyribonucleoside triphosphate,” and “dATP”, “dCTP”, “dGTP”, “dTTP”, and “dUTP” represent the triphosphate derivatives of the individual deoxyribonucleosides.

The term “nucleotide analog” refers to a modified or non-naturally occurring nucleotide, particularly nucleotide analogs which can be polymerized into a nucleic acid sequence, with or without naturally occurring nucleotides, by template directed DNA synthesis. A nucleotide analog may include a modified nucleobase, a modified sugar, a modified phosphate, or a combination of any two or more such modifications. Particular nucleotide analogs are capable of Watson-Crick pairing via hydrogen bonds with a complementary nucleotide and illustratively include, but are not limited to, those containing an analog of a nucleotide base such as substituted purines or pyrimidines, deazapurines, methylpurines, methylpyrimidines, aminopurines, aminopyrimidines, thiopurines, thiopyrimidines, indoles, pyrroles, 7-deazaguanine, 7-deazaadenine, 7-methylguanine, hypoxanthine, pseudocytosine, pseudoisocytosine, isocytosine, isoguanine, 2-thiopyrimidines, 4-thiothymine, 6-thioguanine, nitropyrrole, nitroindole, and 4-methylindole. Nucleotide analogs include those containing an analog of a deoxyribose such as a substituted deoxyribose, a substituted or non-substituted arabinose, a substituted or non-substituted xylose, and a substituted or non-substituted pyranose. Nucleotide analogs include those containing an analog of a phosphate ester such as phosphorothioates, phosphorodithioates, phosphoroamidates, phosphoroselenoates, phosophoroanilothioates, phosphoroanilidates, phosphoroamidates, boronophosphates, phosphotriesters, and alkylphosphonates such as methylphosphonates. Nucleotide analogs include, but are not limited to, inosine, isoG, IsoC, deaza G, deaza A, hypoxanthine, xanthine, 7-methylguanine, inosine, xanthinosine, 7-methylguanosine, 5,6-dihydrouracil, 5-methylcytosine, pseudouridine, dihydrouridine, 5-methylcytidine, dideoxynucleotides, 5-methyl dC, 2,6-diaminopurine, propynyl-deoxyuridine, or 5-hydroxybutynl-2′-deoxyuridine, or any two or more of these or other nucleotide analogs, according to aspects of the present disclosure. Nucleotide analogs include locked nucleic acid monomers, bridged nucleic acid monomers, and peptide nucleic acid monomers.

According to aspects of the present disclosure, the reaction mixture includes dNTPs, with or without one or more nucleotide analogs, in an amount in the range of about pH 0.1 mM to about pH 10 mM, such as about 0.5 mM, about 1 mM, about 2 mM, about 3 mM, about 4 mM, about 5 mM, about 6 mM, about 7 mM, about 8 mM, about 9 mM, about 10 mM, or more, or less.

According to aspects of the present disclosure, the reaction mixture includes a reaction buffer. According to aspects of the present disclosure, the pH range of a reaction buffer is about pH 7.0 to about pH 9.0. According to aspects of the present disclosure, the pH of a reaction buffer is about pH 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, or more, or less.

Any reaction buffer compatible with the reagents and reaction can be used, illustratively including one or more of: sodium phosphate buffer, potassium phosphate buffer, Tris-HCl buffer, Tris-Acetate buffer (Tris-Ac) and Tricine buffer.

According to aspects of the present disclosure, the reaction buffer includes a Tris buffer, such as Tris-HCl, Tricine, or Tris-Ac. According to aspects of the present disclosure, the reaction buffer comprises about 10 mM to about 100 mM Tris-HCl, Tricine, or Tris-Ac. According to aspects of the present disclosure, the reaction buffer comprises about 10 mM to about 100 mM Tris-HCl. According to aspects of the present disclosure, the reaction buffer comprises 10-100 mM Tris-Ac. According to aspects of the present disclosure, the reaction buffer comprises a mixture of both Tris-HCl and Tris-Ac, at the same or different concentrations.

According to aspects of the present disclosure, the reaction buffer comprises Tris-HCl. According to aspects of the present disclosure, the reaction buffer comprises about 10 mM Tris-HCl. According to aspects of the present disclosure, the reaction buffer comprises about 20 mM Tris-HCl. According to aspects of the present disclosure, the reaction buffer comprises about 40 mM Tris-HCl. According to aspects of the present disclosure, the reaction buffer comprises about 50 mM Tris-HCl. According to aspects of the present disclosure, the reaction buffer comprises about 75 mM Tris-HCl. According to aspects of the present disclosure, the reaction buffer comprises about 100 mM Tris-HCl.

According to aspects of the present disclosure, a DNA polymerase included in the reaction mixture is a strand-displacing DNA polymerase. According to aspects of the present disclosure, a DNA polymerase included in the reaction mixture is a strand-displacing DNA polymerase which lacks a 5′ to 3′ exonuclease activity. Illustrative examples of DNA polymerases included in a LAMP reaction mixture according to aspects of the present disclosure include Bacillus stearothermophilus, Bst, DNA polymerase large fragment; Phi29 DNA polymerase; Bacillus smithii, Bsm, DNA polymerase; Geobacillus sp. M (GspM) polymerase; Thermodesulfatator indicus (Tin) polymerase; Taq polymerase Thermus, thermophilus, Tth, DNA polymerase; Thermococcus litoralis DNA polymerase; Pyrococcus furiosus, Pfu, DNA polymerase; Bacillus caldotenax DNA polymerase; Polymerase I Klenow fragment; Bacillus subtilis (Bsu) Pol I polymerase; and may be a wild-type, mutant, or a modified version of any of these or other polymerases having the same or similar activity.

According to aspects of the present disclosure, a LAMP reaction mixture contains one or more additional components, such as, but not limited to, dithiothreitol; a crowding agent, such as polyethylene glycol (PEG), dextran, Ficoll, polyvinyl alcohol, or polyvinyl pyrrolidone; and/or a detergent or ionic or non-ionic surfactant such as, but not limited to, Triton™ X, and SDS.

According to aspects of the present disclosure, an amount of DNA polymerase included in a reaction mixture is in the range of about 0.1 Unit/ul reaction mixture to about 200 Units/ul reaction mixture, such as about 0.1 Units/ul reaction mixture to about 100 Units/ul reaction mixture, such as about 0.5 Units, about 1 Unit, about 5 Units, about 10 Units, about 15 Units, about 20 Units, about 25 Units, about 30 Units, about 35 Units, about 40 Units, about 45 Units, about 50 Units, about 55 Units, about 60 Units, about 65 Units, about 70 Units, about 75 Units, about 80 Units, about 85 Units, about 90 Units, about 95 Units, about 100 Units, or more, or less, per ul of reaction mixture. As used herein, one unit (“U”) of DNA polymerase is defined as the amount of enzyme that will incorporate 10 nmol of dNTP into acid insoluble material in 30 minutes at a specified temperature, such as 65° C.

A LAMP reaction mixture may be present in a container configured to contain the LAMP reaction mixture. Such a container may be a well, tube, plate, depression, channel, slide, droplet, or any other container functional to contain the reaction mixture.

LAMP amplification conditions include incubating the LAMP reaction mixture at a suitable temperature for a suitable period of time to produce a LAMP reaction product comprising LAMP amplified target nucleic acids.

According to aspects of the present disclosure, the LAMP reaction mixture is incubated at a temperature in the range of about 50° C. to about 70° C., such as at 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., or higher, or lower. According to aspects of the present disclosure, the LAMP reaction mixture is incubated at a temperature lower than 50° C., such as at room temperature, typically about 20° C. to about 28° C., or at or near human body temperature, typically about 37° C.

According to aspects of the present disclosure, the LAMP reaction mixture is incubated for a time in the range of about 5 minutes to about 24 hours, such as about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 75 minutes, about 90 minutes, about 120 minutes, about 3 hours, about 4 hours, about 5 hours, about 6 hours, about 7 hours, about 8 hours, about 9 hours, about 10 hours, about 11 hours, about 12 hours, about 24 hours, or longer, or shorter.

A sample to be assayed for a target nucleic acid can be any biological or environmental sample that includes, or may include, nucleic acids.

According to aspects of the present disclosure, the sample is, or is derived from, a biological sample obtained from a subject. A biological sample obtained from a subject can be a bodily fluid, cells, or tissue, such as but is not limited to, a sample of saliva, blood, plasma, serum, mucus, urine, feces, nasal material, cerebrospinal fluid, cerebroventricular fluid, pleural fluids, pulmonary and bronchial lavage samples, bile, sweat, tears, semen, sweat, bladder wash samples, amniotic fluid, lymph, hair, skin, tumor, biopsy material, and peritoneal fluid.

A subject from which a sample is obtained can be any type of organism including, but not limited to, a mammal such as a human; a non-human primate; a rodent such as a mouse, rat, or guinea pig; a domesticated pet such as a cat or dog; a horse, cow, pig, sheep, goat, or rabbit; a bird, a reptile, an amphibian, an insect, bacteria, protozoa, a plant, or a virus. Subjects can be either gender and can be any age. In aspects of methods of the present disclosure, the subject is human.

According to aspects of the present disclosure, the sample is, or is derived from, an environmental sample. An environmental sample can be, but is not limited to, a liquid, gas, or solid sample, including, but not limited to, a water sample, a sewage sample, an air sample, a surface swab, a food sample, a beverage sample, a clothing sample, and a soil sample. According to aspects of the present disclosure, the sample is a sewage sample.

More than one target nucleic acid may be assayed in a LAMP reaction according to aspects of the present disclosure, i.e. multiplex LAMP reactions. According to aspects of the present disclosure, more than one target nucleic acid from a single sample may be assayed in a LAMP reaction. According to aspects of the present disclosure, more than one target nucleic acid may be assayed in a LAMP reaction wherein the target nucleic acids are from more than one sample.

The LAMP amplified target nucleic acids, i.e. the LAMP reaction products, are detected according to aspects of the present disclosure.

Any of various modalities for detecting the LAMP amplified target nucleic acids can be used in methods of the present disclosure including, but not limited to, one or more of: pH, turbidity, an electrophoresis pattern, and a detectable label.

LAMP reactions are characterized by decreasing pH as the reaction proceed. Therefore, detection of pH change, such as by measurement of pH, or by assessment of a pH-dependent indicator, such as by eye or by color detection instrumentation, can be used to detect production of LAMP amplified target nucleic acids. pH-sensitive indicators include, for example, alizarin yellow, azolitmin, bromocresol green, bromocresol violet, bromophenol blue, cresol red; methyl orange, methyl red, methyl yellow, naphtholphthalein; naphthyl red, neutral red; nile blue, nitramine, phenolphthalein; phenol red; salicyl yellow, thymol blue; 5-(and-6)carboxy SNARF-1, and thymolphthalein.

A change in turbidity occurs during LAMP reactions due to production of LAMP amplified target nucleic acids. Increased turbidity can be detected, such as by eye or by turbidimetry.

According to aspects of the present disclosure, detecting the amplified target nucleic acids comprises detecting a detectable label.

The terms “detectably labeled” and “detectable label” refer to a material capable of producing a signal indicative of the presence of a LAMP amplified target nucleic acid and detectable by any appropriate method illustratively including spectroscopic, optical, photochemical, biochemical, enzymatic, electrical and/or immunochemical. A detectable label allows for detection based on detectable properties of the label, such as, but not limited to, chemical properties, electrical properties, magnetic properties, optical properties, physical properties, or any two or more thereof. The detectable label may include one or more of: a fluorescent label, a bioluminescent label, a chemiluminescent label, a chromophore, a magnetic label, an antibody, an antigen, an enzyme, a substrate, a radioisotope, or any two or more thereof.

Fluorophores used as fluorescent labels can be any of numerous fluorophores including, but not limited to, those described in Haughland, R. P., The Handbook, A Guide to Fluorescent Probes and Labeling Technologies, 10th Ed., 2005; Lakowicz, J. R., Principles of Fluorescence Spectroscopy, Springer, 3rd ed., 2006; 4-acetamido-4′-isothiocyanatostilbene-2,2′disulfonic acid; acridine and derivatives such as acridine and acridine isothiocyanate; 4-amino-N-[3-vinylsulfonyl)phenyl]naphthalimide-3,5 disulfonate, Lucifer Yellow VS; N-(4-anilino-1-naphthyl)maleimide; anthranilamide, Brilliant Yellow; BIODIPY fluorophores (4,4-difluoro-4-bora-3a,4a-diaza-s-indacenes); coumarin and derivatives such as coumarin, 7-amino-4-methylcoumarin (AMC, Coumarin 120), 7-amino-4-trifluoromethylcouluarin (Coumaran 151); cyanine dyes; cyanosine; DAPDXYL sulfonyl chloride; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylaminolnaphthalene-1-sulfonyl chloride (DNS, dansyl chloride); 4-4′-dimethylaminophenylazo)benzoic acid (DABCYL); 4-dimethylaminophenylazophenyl-4′-isothiocyanate (DABITC); EDANS (5-[(2-aminoethyl)amino]naphthalene-1-sulfonic acid), eosin and derivatives such as eosin isothiocyanate; erythrosin and derivatives such as erythrosin B and erythrosin isothiocyanate; ethidium such as ethidium bromide; fluorescein and derivatives such as 5-carboxyfluorescein (FAM), 6-carboxyfluorescein (6-FAM); hexachlorofluorescenin, 5-(4,6-dichlorotriazin-2-yl)aminofluorescein (DTAF), 2′,7′-dimethoxy-4′,5′-dichloro-6-carboxyfluorescein (JOE) and fluorescein isothiocyanate (FITC); fluorescamine; green fluorescent protein and derivatives such as EBFP, EBFP2, ECFP, and YFP; IAEDANS (5-({2-[(iodoacetyl)amino]ethyl}amino)naphthalene-1-sulfonic acid), Malachite Green isothiocyanate; 4-methylumbelliferone; orthocresolphthalein; nitrotyrosine; pararosaniline; Phenol Red; B-phycoerytnin; o-phthaldialdehyde; pyrene and derivatives such as pyrene butyrate, 1-pyrenesulfonyl chloride and succinimidyl 1-pyrene butyrate; QSY 7; QSY 9; Reactive Red 4 (Cibacron® Brilliant Red 3B-A); rhodamine and derivatives such as 6-carboxy-X-rhodamine (ROX), 6-carboxyrhodamine (Rhodamine 6G), rhodamine isothiocyanate, lissamine rhodamine B sulfonyl chloride, rhodamine B, rhodamine 123, sulforhodamine B, sulforhodamine 101 and sulfonyl chloride derivative of sulforhodamine 101 (Texas Red); N,N,N′,N-tetramethyl-carboxyrhodamine (TAMRA); tetramethyl rhodamine; tetramethyl rhodamine isothiocyanate (TRITC); riboflavin; rosolic acid, terbium chelate derivatives, and xanthenes.

According to aspects of the present disclosure, detecting the amplified target nucleic acids includes detecting a fluorescent emission signal of a fluorescent intercalating dye.

According to aspects of the present disclosure, a detectable label is present in one or more primers of the LAMP assay primer set.

According to aspects of the present disclosure, a detectable label is a fluorescent label present in one or more primers of the LAMP assay primer set, incorporated into the amplified target nucleic acids.

According to aspects of the present disclosure, detecting the amplified target nucleic acids includes detecting a fluorescence resonance energy transfer (FRET) fluorescent label.

According to aspects of the present disclosure, a detectable label is a fluorescent label present in a probe, and wherein the probe is a sequence-specific binding partner for a specified sequence present in the amplified target nucleic acids. Such a probe used to detect a LAMP amplification product may be called a “molecular beacon.”

The term “molecular beacon” refers to a molecule which is conditionally detectable. A molecular beacon which is a probe includes a nucleic acid sequence which is a sequence-specific binding partner for a specified sequence present in the LAMP amplified target nucleic acids, i.e. LAMP product.

According to aspects of the present disclosure, the probe is a fluorescence resonance energy transfer (FRET)-labeled oligonucleotide probe, wherein the FRET label includes a quencher and a fluorescent moiety such that the quencher quenches a fluorescent signal of the fluorescent moiety when the oligonucleotide probe is in stem-loop configuration and not hybridized to the specified sequence, and wherein the fluorescent signal of the fluorescent moiety is detectable when the fluorescently-labeled oligonucleotide probe is specifically hybridized to the specified sequence.

A molecular beacon probe used in methods according to aspects of the present disclosure includes a fluorophore which is a FRET donor, and a fluorescence quencher which is a FRET acceptor. The molecular beacon probe is configured such that fluorescent emission of the fluorophore is quenched by the quencher unless the molecular beacon probe is specifically hybridized to its sequence-specific binding partner, i.e. specified sequence present in the LAMP amplified target nucleic acids, i.e. the LAMP product. When the molecular beacon probe is specifically hybridized to the specified sequence present in the LAMP product, fluorescence emission of the fluorophore is detectable.

A molecular beacon probe may be labeled with a fluorophore, a FRET donor, at or near the 5′ end of the probe and a fluorescence quencher, a FRET acceptor, at or near the 3′ end of the probe, or vice versa, such that when the nucleic acid sequence is in hairpin form, the fluorophore and quencher are in proximity so that substantially no fluorescent signal is detectable from the fluorophore.

According to aspects of the present disclosure, a molecular beacon probe is configured to have a size and nucleic acid sequence which allows the probe to form a hairpin when not specifically hybridized to the specified sequence present in the LAMP product.

By contrast, when the molecular beacon probe is specifically hybridized to the specified sequence present in the LAMP product, it is not in hairpin form such that the fluorophore is no sufficiently in proximity for the fluorescence quencher to inhibit fluorescence emission by the FRET donor fluorophore, resulting in a fluorescent signal emitted by the FRET donor fluorophore.

Typically, a molecular beacon probe include an oligonucleotide having a length in the range of 15 to 35 contiguous nucleotides in length, such as 16 to 30 contiguous nucleotides in length, such as 18 to 25 contiguous nucleotides in length, and may be shorter or longer. Typically, a FRET donor is attached to the molecular beacon probe and is present at or near the 5′ end of a molecular beacon probe and a FRET acceptor is attached to the molecular beacon probe and is present at or near the 3′ end of a molecular beacon probe, separated by a distance of about 20 to 30 nucleotides, although a greater or lesser distance is possible as long as the quenching activity of the quencher is capable of functioning at the selected distance.

A molecular beacon probe may include one or more nucleotide analogs which increase the Tm of the probe. Such a probe may be, for example, a locked nucleic acid sequence by incorporation of locked nucleic acid monomer(s).

Fluorophores included as labels according to aspects of the present disclosure, include FRET donors such as, but not limited to, 4-acetamido-4′-isothiocyanatostilbene-2,2′disulfonic acid; acridine and derivatives such as acridine and acridine isothiocyanate; 4-amino-N-[3-vinylsulfonyl)phenyl]naphthalimide-3,5 disulfonate, Lucifer Yellow VS; N-(4-anilino-1-naphthyl)maleimide; anthranilamide, Brilliant Yellow; BIODIPY fluorophores (4,4-difluoro-4-bora-3a,4a-diaza-s-indacenes); coumarin and derivatives such as coumarin, 7-amino-4-methylcoumarin (AMC, Coumarin 120), 7-amino-4-trifluoromethylcouluarin (Coumaran 151); Cy5, Cy7, cyanosine; DAPDXYL sulfonyl chloride; 4′,6-diaminidino-2-phenylindole (DAPI); 5′,5″-dibromopyrogallol-sulfonephthalein (Bromopyrogallol Red); 7-di ethyl amino-3-(4′-isothiocyanatophenyl)-4-methylcoumarin; diethylenetriamine pentaacetate; 4,4′-diisothiocyanatodihydro-stilbene-2,2′-di sulfonic acid; 4,4′-diisothiocyanatostilbene-2,2′-disulfonic acid; 5-[dimethylaminolnaphthalene-1-sulfonyl chloride (DNS, dansyl chloride); 4-dimethylaminophenylazophenyl-4′-isothiocyanate (DABITC); EDANS (5-[(2-aminoethyl)amino]naphthalene-1-sulfonic acid), eosin and derivatives such as eosin isothiocyanate; erythrosin and derivatives such as erythrosin B and erythrosin isothiocyanate; ethidium such as ethidium bromide; fluorescein and derivatives such as 5-carboxyfluorescein (FAM), 5-(4,6-dichlorotriazin-2-yl)aminofluorescein (DTAF), 2′,7′-dimethoxy-4′,5′-dichloro-6-carboxyfluorescein (JOE), hexachlorofluorescein (HEX), tetramethyl fluorescein (TET), and fluorescein isothiocyanate (FITC); fluorescamine; IAEDANS (5-({2-[(iodoacetyl)amino]ethyl}amino)naphthalene-1-sulfonic acid), Malachite Green isothiocyanate; 4-methylumbelliferone; NED, orthocresolphthalein; nitrotyrosine; pararosaniline; Phenol Red; B-phycoerytnin; o-phthaldialdehyde; pyrene and derivatives such as pyrene butyrate, 1-pyrenesulfonyl chloride and succinimidyl 1-pyrene butyrate; Reactive Red 4 (Cibacron® Brilliant Red 3B-A); rhodamine and derivatives such as 6-carboxy-X-rhodamine (ROX), 6-carboxyrhodamine (Rhodamine 6G), rhodamine isothiocyanate, lissamine rhodamine B sulfonyl chloride, rhodamine B, rhodamine 123, sulforhodamine B, sulforhodamine 101 and sulfonyl chloride derivative of sulforhodamine 101 (Texas Red); tetramethyl rhodamine; N,N,N′,N-tetramethyl-carboxyrhodamine (TAMRA); tetramethyl rhodamine isothiocyanate (TRITC); TEX 615, VIC, and Yakima Yellow.

According to aspects of the present disclosure, the fluorophore is a fluorescent polymeric dye, such as, but not limited to, brilliant violet fluorophores, see for example Chattopadhyay et al., Brilliant violet fluorophores: A new class of ultrabright fluorescent compounds for immunofluorescence experiments, Cytometry Part A, 81A(6):456-466, 2012.

Quenchers useful in methods according to aspects of the present disclosure are FRET acceptors such as, but not limited to, BHQ1, BHQ2, BHQ3, tetramethylrhodamine, N,N,N′,N-tetramethyl-carboxyrhodamine (TAMRA); fluorescein, 4-(4′-dimethylaminophenylazo)benzoic acid (DABCYL), BODIPY FL, QSY 7, QSY 9, and Alexa647.

Optionally, an internal quencher can be used to supplement the activity of a FRET pair including a FRET donor present at or near the 5′ end of a molecular beacon probe and a FRET acceptor present at or near the 3′ end of a molecular beacon probe. An internal quencher is attached to the molecular beacon probe between the FRET donor present at or near the 5′ end of a molecular beacon probe and a FRET acceptor is present at or near the 3′ end of a molecular beacon probe. An internal quencher can provide greater overall quenching of the FRET donor, lower background, and increase signal detection. Internal quenchers include, but are not limited to, the ZEN internal quencher and the TAO internal quencher.

One of skill in the art can easily determine which of various fluorophores and quenchers are to be used as FRET donor/acceptor pairs in a particular application.

Examples of FRET donor/acceptor pairs are described in Lakowicz, J. R., Principles of Fluorescence Spectroscopy, Springer, 3rd ed., 2006; and Haughland, R. P., The Handbook, A Guide to Fluorescent Probes and Labeling Technologies, 10th Ed., 2005.

One of skill in the art can easily determine which of various fluorophores and quenchers are to be used as FRET donor/acceptor pairs in a particular application.

Fluorophores are selected for inclusion as a detectable label, such as in molecular beacon probes, based on characteristics including, but not limited to, excitation maximum wavelength and emission maximum wavelength.

Attachment of detectable label to a nucleic acid sequence, such as a fluorophore and/or quencher, can be by direct coupling to the nucleic acid or indirect, such as by an intervening spacer. A detectable label can be incorporated into a nucleic acid by any of various well-known methods such as by introduction of a detectable label-modified base into an oligonucleotide. Methods suitable for attachment of detectable label to an oligonucleotide are exemplified in Nucleic Acids Res., 25: 2923-2929, 1997 and WO/2005/051967.

According to aspects of the present disclosure, a fluorescent label may be an intercalating dye. Intercalating dyes include, but are not limited to, ethidium bromide, Syto-9, Syto-82, SYBR green, propidium iodide, YOYO-1, and DAPI.

According to aspects of the present disclosure, two or more reaction mixtures are included in single container, i.e. multiplexed, such as a reaction vessel, for example using distinguishable fluorophores.

For multiplex reactions according to aspects of the present disclosure, at least two fluorophores are selected such that their emission maxima are detectably different, allowing for detection of fluorescence from the at least two fluorophores, thereby providing a separate signal from the at least two individual fluorophores when the at least two fluorophores are excited simultaneously, or at different times present in a single reaction mixture, or in separate reaction mixtures.

According to aspects of the present disclosure, two, three, four, five, six, seven, or more, fluorophores are selected such that their emission maxima are detectably different, allowing for simultaneous detection of fluorescence from the fluorophores, thereby providing a separate signal from the individual fluorophores when the fluorophores are excited simultaneously, or at different times, present in a single reaction mixture, or in separate reaction mixtures.

According to aspects of the present disclosure, the probe is a molecular beacon probe which is a sequence-specific binding partner for a specified sequence present in the forward strand of the amplified target nucleic acids when FIP has a molar concentration at least 50% higher than the molar concentration of BIP and no more than 300% higher than the molar concentration of BIP.

According to aspects of the present disclosure, the probe is a molecular beacon probe which is a sequence-specific binding partner for a specified sequence present in the backward strand of the amplified target nucleic acids when BIP has a molar concentration at least 50% higher than the molar concentration of FIP and no more than 300% higher than the molar concentration of FIP.

The term “nucleic acid” as used herein refers to RNA or DNA molecules having more than one nucleotide in any form including single-stranded, double-stranded, oligonucleotide or polynucleotide. The term “nucleotide sequence” is used to refer to the ordering of nucleotides in an oligonucleotide or polynucleotide in a single-stranded form of nucleic acid.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100% complementary to a region of the nucleotide sequence 5′-TTAGCTGG-3′.

The terms “hybridization” and “hybridizes” refer to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

The terms “specific hybridization,” “specifically hybridizes” and grammatical equivalents refer to hybridization of a particular nucleic acid to a target nucleic acid without substantial hybridization to nucleic acids other than the target nucleic acid in a sample.

Embodiments of inventive compositions and methods are illustrated in the following examples. These examples are provided for illustrative purposes and are not considered limitations on the scope of inventive compositions and methods.

### EXAMPLES

In these Examples, described and shown in text and figures herein, the following definitions, acronyms, and abbreviations are used:
dsDNA—Double stranded DNA (deoxyribonucleic acid); ssDNA—Single stranded DNA; qPCR—Quantitative real time PCR; LAMP—Loop-mediated isothermal amplification; IAC—Internal Amplification Control; RT—Room Temperature; Tth—Time to Threshold; LOD—Limit of Detection; NTC—Negative Control Without Target Organism Template

In these Examples, Primer Mix A, Primer Mix B, or Primer Mix C as set forth in Tables 1, 2, and 3, respectively, or modified as described in the individual examples, was used as indicated in the individual Examples:

TABLE 1
Primer Mix A

| | Final |
|---|---|
| Primer | Concentration (μM) |
| [Nuclease-Free Water] | — |
| FIP | 1.6 |
| BIP | 1.6 |
| F3 | 0.2 |
| B3 | 0.2 |
| LF | 0.4 |
| LB | 0.4 |
| Molecular Beacon - | 0.2 |
| Backwards | |

TABLE 2
Primer Mix B

| | Final |
|---|---|
| Primer | Concentration (μM) |
| [Nuclease-Free Water] | — |
| FIP | 0.6 |
| BIP | 1.4 |
| F3 | 0.2 |
| B3 | 0.2 |
| LF | 1.2 |
| LB | 0.6 |
| Molecular Beacon - | 0.2 |
| Backwards | |

TABLE 3
Primer Mix C

| | Final |
|---|---|
| Primer | Concentration (μM) |
| [Nuclease-Free Water] | — |
| FIP | 2.0 |
| BIP | 3.0 |
| F3 | 1.0 |
| B3 | 0.2 |
| LF | 2.2 |
| LB | 0.6 |
| Molecular Beacon - | 0.2 |
| Backwards | |

Primer stocks are stored in lyophilized form and are then reconstituted with nuclease-free water for use.

Reagents used in these Examples, included: Bst DNA Polymerase; Large-Fragment, Glycerol-Free (NewEngland BioLabs); Lyo-Ready Bst Mix Prototype C (Meridian Bioscience); 10×PCR Buffer II (Molecular Cloning Laboratories); Magnesium Sulfate Heptahydrate (Sigma-Aldrich); Nuclease-Free Water (Integrated DNA Technologies); dNTPs (Meridian Bioscience); Tris-EDTA Buffer (Sigma-Aldrich); Syto-9 Nucleic Acid stain (ThermoFisher); and others specified in each Example.

For Negative Template Controls, the volume of Genomic DNA Template is substituted with Tris-EDTA Buffer (Sigma-Aldrich).

Note that each separate stock of dNTP was provided individually at 100 mM. These stocks of dATP, dCTP, dTTP, and dGTP were combined in a ratio of 1:1:1:1 to create a 25 mM/EA dNTP stock.

TABLE 4
Single-plex or Duplex Target + Template Master Mix − Formulation

| | | | | | | | |
|---|---|---|---|---|---|---|---|
| | | | | Vol. per | | | Total |
| | | | | 25 μL | | Vol. per | MM |
| # of | # of | R × N | | Reaction | Final | Condition | Vol. |
| Conditions | Replicates | Overage | Reagent | (μL) | Concentration | (μL) | (μL) |

| | | | | | | | |
|---|---|---|---|---|---|---|---|
| 3 | 3 | 25% | [Nuclease- | 14.50 | — | 54.4 | 163.1 |
| | | | Free Water] | | | | |
| | | | 4× PCR | 6.25 | 1× | 23.4 | 70.3 |
| | | | Buffer | | | | |
| | | | 20× Primer | 1.00 | 1× | 3.8 | 11.3 |
| | | | Mix #1 | | | | |
| | | | 20× Primer | 1.25 | 1× | 4.7 | 14.1 |
| | | | Mix #2 | | | | |
| | | | (Optional) | | | | |
| | | | 10× Target | 1.00 | varies | 3.8 | — |
| | | | Template #1 | | | | |
| | | | 10× Target | 1.00 | varies | 3.8 | — |
| | | | Template #2 | | | | |
| | | | (Optional) | | | | |

| | | |
|---|---|---|
| Total Reaction Volume | 25.0 | 86.3 |

TABLE 5

| | | | | |
|---|---|---|---|---|
| Split Master Mix into Aliquots of (μL): | 86.3 | across | 3 | tube(s) |

| | | |
|---|---|---|
| Into Each Aliquot Add (μL): | 3.8 | Template #1 |
| Into Each Aliquot Add (μL): | 3.8 | Template #2 (Optional) |

For Single-Plex (One Template) Wet LAMP Reactions

LAMP reaction mixtures are prepared including the components indicated. The nucleic acid template is added last, after the master mixes have been aliquoted into separate tubes for the appropriate test conditions, bringing the total reaction volume to 25 micoliters. The tubes are then mixed thoroughly, followed by micro-centrifuging the tubes at 10 k rpm briefly. Transfer the final 25 uL wet LAMP reaction mixture into wells of an opaque white 96-well microtiter plate. Each plate was sealed and loaded into a plate-spinner for about 1 minute of centrifugation so that the liquid settled onto the bottom of each well of the plate. The plates are inserted into a qPCR instrument programmed for a 20-minute isothermal amplification step at 60° C., followed by a 5 minute ramp-down that cools at −2° C. per 30-second cycle.

Following the LAMP reaction, the fluorescence detection capability of the qPCR instrument is used to detect fluorescence from the Molecular Beacon probe in each well of the plates.

For Duplex Wet LAMP Reactions

LAMP reaction mixtures are prepared including the components indicated, including two nucleic acid target templates. The nucleic acid templates are added last, after the master mixes have been aliquoted into separate tubes for the appropriate test conditions, bringing the total reaction volume to 25 micoliters. The tubes are then mixed thoroughly, followed by micro-centrifuging the tubes at 10 k rpm briefly. Transfer the final 25 uL wet LAMP reaction mixture into wells of an opaque white 96-well microtiter plate. Each plate was sealed and loaded into a plate-spinner for about 1 minute of centrifugation so that the liquid settled onto the bottom of each well of the plate. The plates are inserted into a qPCR instrument programmed for a 20-minute isothermal amplification step at 60° C., followed by a 5 minute ramp-down that cools at −2° C. per 30-second cycle.

Following the LAMP reaction, the fluorescence detection capability of the qPCR instrument is used to detect fluorescence from the Molecular Beacon probe in each well of the plates.

### Example 1

In this Example, the following primers were used:

F3 primer
(SEQ ID NO: 1)
5′-GAACGTGTCGCGGAAGTC-3′
B3 primer
(SEQ ID NO: 2)
5′-CGGCAATAGCGTCACCTT-3′
FIP primer
(SEQ ID NO: 3)
5′-GCGCGGCATCCGCATCAATATCTGGATGGTATGCCCGG-3′
BIP primer
(SEQ ID NO: 4)
5′-GCGAACGGCGAAGCGTACTGTCGCACCGTCAAAGGAAC-3′
LoopF primer
(SEQ ID NO: 5)
5′-TCAAATCGGCATCAATACTCATCTG-3′
LoopB primer
(SEQ ID NO: 6)
5′-AAAGGGAAAGCCAGCTTTACG-3′

In this example, the target was a portion of a nucleic acid encoding the Invasion A protein of Salmonella typhimurium. The target sequence was:

(SEQ ID NO: 7)
5′GAACGTGTCGCGGAAGTCGCGGCCCGATTTTCTCTGGATGGTATGCC
CGGTAAACAGATGAGTATTGATGCCGATTTGAAGGCCGGTATTATTGAT
GCGGATGCCGCGCGCGAACGGCGAAGCGTACTGGAAAGGGAAAGCCAGC
TTTACGGTTCCTTTGACGGTGCGATGAAGTTTATCAAAGGTGACGCTAT
TGCCG-3′

The Molecular Beacon probe used in this example has the sequence:

(SEQ ID NO: 8)
5′-CGTAAAGGGAAAGCCAGCTTTACG-3′

The underlined portion of the molecular beacon sequence hybridizes with a region of the Invasion A gene sequence corresponding to a portion of the LB primer. A fluorophore and quencher were attached to the oligonucleotide of the molecular beacon, the fluorophore was FAM (6-carboxyfluorescein) and the quencher was BHQ1 (Black Hole Quencher 1).

In this example, the LAMP reaction to detect the target is carried out in a 25 μl volume (total) containing the following components: Primer Mix A or Primer Mix B, 6 mM MgSO₄, 1.4 mM of each dNTP, 1× isothermal amplification reaction buffer (where a 10× solution is composed of 200 mM of Tris-HCl pH 8.8, 100 mM of (NH₄)₂SO₄, 100 mM of KCl, 20 mM MgSO₄, and 1% Tween-20), 8 Units of Bst Wild-Type Large Fragment polymerase (New England BioLabs), and at least 100 copies of target DNA. The reaction mixture is made by combining the reagents as indicated in Tables 4 and 5.

The LAMP amplification is carried out using the Bio-Rad CFX96 Touch Real-Time PCR Detection System (Hercules, California, USA). The reaction mixture is heated at 60° C. for 20 minutes. Negative and positive controls are included in each run.

Detection

Generation of the reaction product was detected using the Bio-Rad CFX96 Touch Real-Time PCR Detection System. 25 μl reactions were aliquoted into single wells of a standard 96-well microtiter plate and placed as per machine manufacturer's instructions into the instrument and into the path of a fluorescent beam using the FAM channel to excite the fluorescent moiety of the molecular beacon probe. A signal in the emission wavelength of the fluorescent moiety was detected. A negative control containing no target DNA template (with the same volume substituted with nuclease-free water in the final master mix) was used.

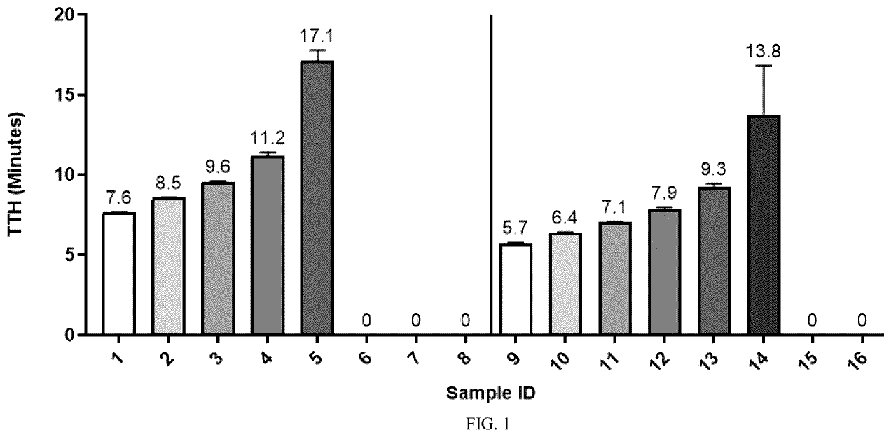

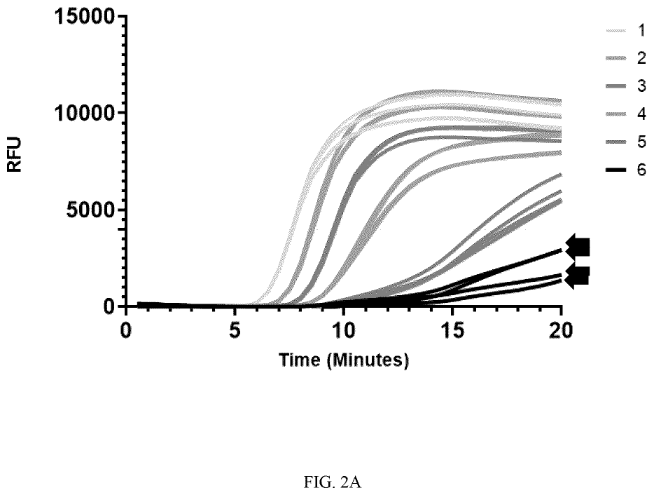

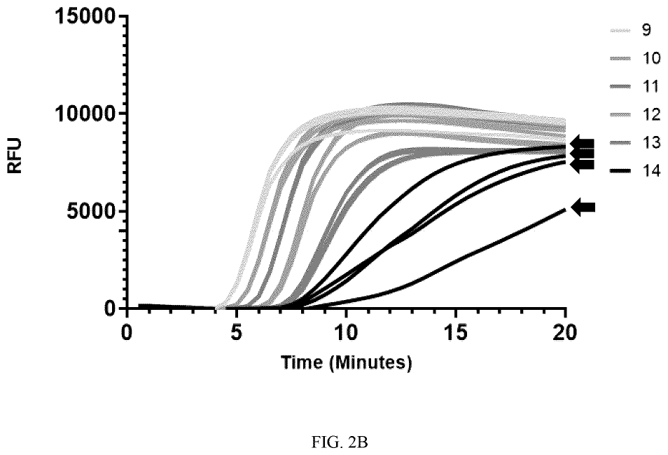

FIG. 1 shows results of LAMP assays in which samples #1-8 were the results using a master mix containing the concentrations as stated in Primer Mix A in Table 1. FIG. 1 also shows results of LAMP assays in which samples #9-16 were the results of a master mix containing the concentrations as stated in Primer Mix B, Table 2. Samples #1-7 and #9-15 were in a multiplex assay format. Samples #8 & 16 were in singleplex format. Samples #1 & #9 contain 10\hat{ }6 copies of the target. Samples #2 & #10 contain 10\hat{ }5 copies of the target. Samples #3 & #11 contain 10\hat{ }4 copies of the target. Samples #4 &12 contain 10\hat{ }3 copies of the target. Samples #5 & #13 contain 10\hat{ }2 copies of the target. Samples #6 & #14 contain 10\hat{ }1 copies of the target. Samples #7, 8, 15, and 16 contain 0 copies of the target. TTH was defined as the time elapsed until the amplification curve reached a minimum threshold height (4000 RFU), shown in FIG. 2A and FIG. 2B.

FIG. 2A is a graph showing the real-time amplification curve output from a dilution series of target genomic DNA at concentrations ranging from 10\hat{ }6 to 10\hat{ }1, samples 1, 2, 3, 4, 5, and 6, respectively. FIG. 2B is a graph showing the real-time amplification curve output from a dilution series of target genomic DNA at concentrations ranging from 10\hat{ }6 to 10\hat{ }1, samples 9, 10, 11, 12, 13, and 14, respectively. The four replicates for Sample #6 and Sample #14 are denoted with the darkest black arrows and darkest black lines in FIGS. 2A and 2B, respectively. The results shown in FIG. 2A and FIG. 2B demonstrate a difference in assay sensitivity between Sample #6 (using the stipulated mix from Primer Mix A) and Sample #14 (using the stipulated mix from Primer Mix B) as is clearly demonstrated by comparison of the darkest black lines in FIG. 2A with the darkest black lines in FIG. 2B. The data presented in FIGS. 2A and 2B was also shown in bar graph format in FIG. 1.

### Example 2

In this Example, the following primers were used:

F3 primer
(SEQ ID NO: 1)
5′-GAACGTGTCGCGGAAGTC-3′
B3 primer
(SEQ ID NO: 2)
5′-CGGCAATAGCGTCACCTT-3′
FIP primer
(SEQ ID NO: 3)
5′-GCGCGGCATCCGCATCAATATCTGGATGGTATGCCCGG-3′
BIP primer
(SEQ ID NO: 4)
5′-GCGAACGGCGAAGCGTACTGTCGCACCGTCAAAGGAAC-3′
LoopF primer
(SEQ ID NO: 5)
5′-TCAAATCGGCATCAATACTCATCTG-3′
LoopB primer
(SEQ ID NO: 6)
5′-AAAGGGAAAGCCAGCTTTACG-3′

In this example, the target was a portion of a nucleic acid encoding the Invasion A protein of Salmonella typhimurium. The target sequence was:

(SEQ ID NO: 7)
5′GAACGTGTCGCGGAAGTCGCGGCCCGATTTTCTCTGGATGGTATGCC
CGGTAAACAGATGAGTATTGATGCCGATTTGAAGGCCGGTATTATTGAT
GCGGATGCCGCGCGCGAACGGCGAAGCGTACTGGAAAGGGAAAGCCAGC
TTTACGGTTCCTTTGACGGTGCGATGAAGTTTATCAAAGGTGACGCTAT
TGCCG-3′

The Molecular Beacon probe used in this example has the sequence:

(SEQ ID NO: 8)
5′-CGTAAAGGGAAAGCCAGCTTTACG-3′

The underlined portion of the molecular beacon sequence hybridizes with a region of the Invasion A gene sequence corresponding to a portion of the LB primer. A fluorophore and quencher were attached to the oligonucleotide of the molecular beacon, the fluorophore was FAM (6-carboxyfluorescein) and the quencher was BHQ1 (Black Hole Quencher 1).

In this example, the LAMP reaction to detect the target is carried out in a 25 μl volume (total) containing the following components: Primer Mix A or Primer Mix B, 6 mM MgSO₄, 1.4 mM of each dNTP, 1× isothermal amplification reaction buffer (where a 10× solution is composed of 200 mM of Tris-HCl pH 8.8, 100 mM of (NH₄)₂SO₄, 100 mM of KCl, 20 mM MgSO₄, and 1% Tween-20), 8 Units of Bst Wild-Type Large Fragment polymerase (New England BioLabs), and at least 100 copies of target DNA. The reaction mixture is made by combining the reagents as indicated in Tables 4 and 5.

The LAMP amplification is carried out using the Bio-Rad CFX96 Touch Real-Time PCR Detection System (Hercules, California, USA). The reaction mixture is heated at 60° C. for 20 minutes. Negative and positive controls are included in each run.

Detection

Generation of the reaction product was detected using the Bio-Rad CFX96 Touch Real-Time PCR Detection System. 25 μl reactions were aliquoted into single wells of a standard 96-well microtiter plate and placed as per machine manufacturer's instructions into the instrument and into the path of a fluorescent beam using the FAM channel to excite the fluorescent moiety of the molecular beacon probe. A signal in the emission wavelength of the fluorescent moiety was detected. A negative control containing no target DNA template (with the same volume substituted with nuclease-free water in the final master mix) was used.

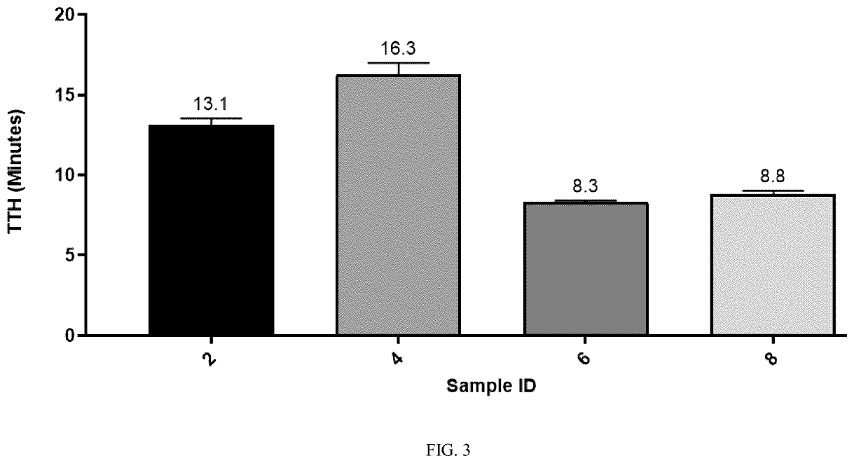

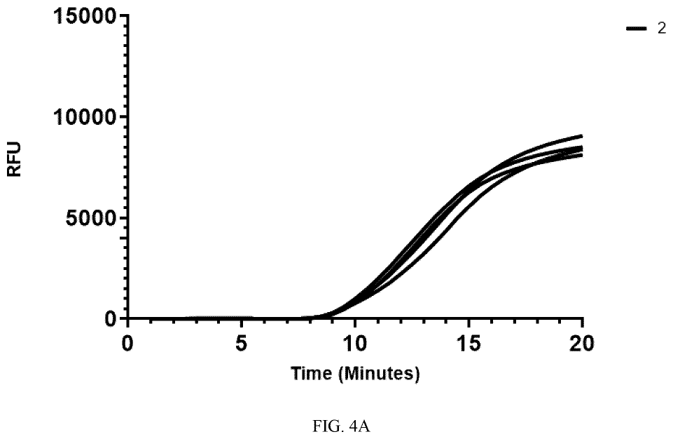

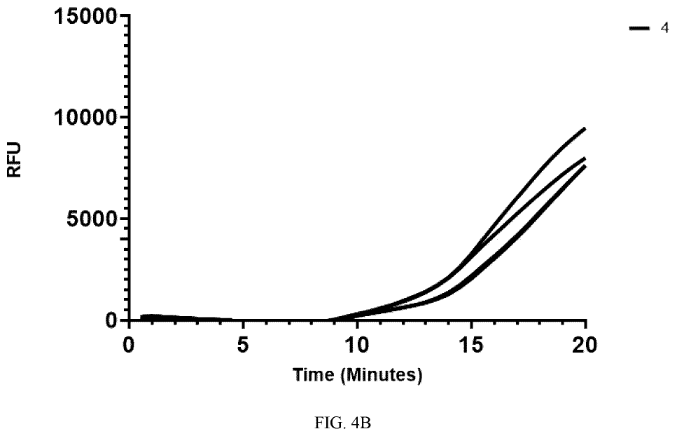

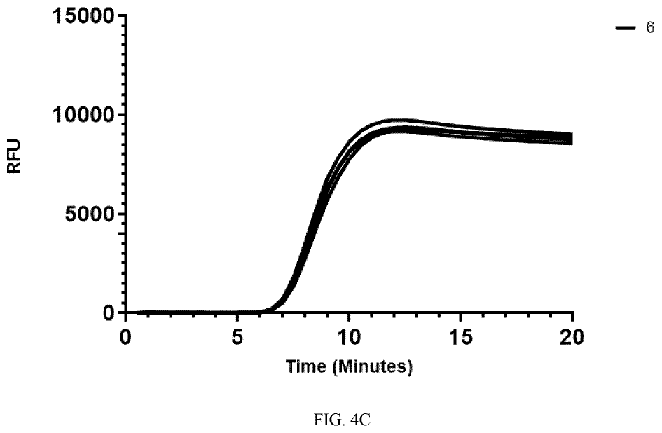

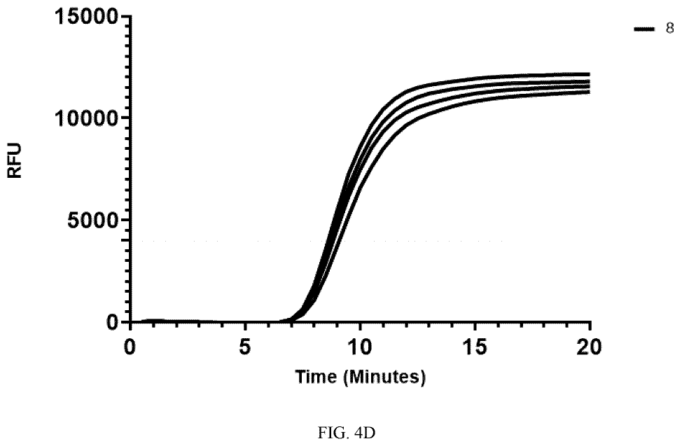

FIG. 3 shows results of LAMP assays in which samples #2 and #4 were obtained using a master mix containing the concentrations stated in Primer Mix A in Table 1. FIG. 3 also shows results of LAMP assays in which samples #6 and #8 were obtained using a master mix containing the concentrations stated in Primer Mix B, Table 2. In addition, Sample #4 and Sample #8 both contained a “Forwards” molecular beacon at the same concentration as the “Backwards” molecular beacon. All samples were tested at a concentration of 10\hat{ }2 copies of target genomic DNA. TTH was defined as the time elapsed until the amplification curve reached a minimum threshold height (4000 RFU), shown in FIGS. 4A, 4B, 4C, and 4D for samples #2, #4, #6, and #8, respectively.

FIGS. 4A, 4B, 4C, and 4D are graphs showing the amplification curves of the corresponding data shown in the graph of FIG. 3.

### Example 3

In this Example, the following primers were used:

F3 primer
(SEQ ID NO: 9)
5′-CGGCGAACAGTAAGGAAG-3′
B3 primer
(SEQ ID NO: 10)
5′-ACTGGCCATGACTGGTAT-3′
FIP primer
(SEQ ID NO: 11)
5′-TGGTGGTTCTGTTAGATCCAACAAGCATCGCGGAATATGG-3′
BIP primer
(SEQ ID NO: 12)
5′-TTCGGCCAACGGCGATCATGAAACTCCTGGTTTATCTG-3′
LoopF primer
(SEQ ID NO: 13)
5′-CAAACCCTACACCATTATCTGT-3′
LoopB primer
(SEQ ID NO: 14)
5′-GCGGCGACATCATTATGGA-3′

In this example, the target was a portion of a nucleic acid Z3276 (GenBank CP015855.1:2933401-2933700) of Escherichia coli strain EDL933-1. The target sequence was:

(SEQ ID NO: 15)
CGGCGAACAGTAAGGAAGGAACAATTACGTTGAAATGTGATAATCTTTT
CGGCGACAAAAAACAAGCATCGCGGAATATGGTTGTATATCTTTCTAGC
AGTGACTTAGTTAAAGGAAGTAATACTATTTTGCGTGGTAAAACAGATA
ATGGTGTAGGGTTTGTGTTGGATCTAACAGAACCACCAAAAGGGACTGA
GGCTGCCATTAAAATTTCGGCCAACGGCGATCAGGGCGCGGCGACATCA
TTATGGAAAACAGATAAACCAGGAGTTTCATTAAATAGCAACATTATTA
ATATACCAGTCATGGCCAGT

Molecular beacon:

(SEQ ID NO: 16)
5′-TCACCATGGGCGCGGCGACATCATTATGGTGA

A fluorophore and quencher were attached to the oligonucleotide of the molecular beacon, the fluorophore was FAM (6-carboxyfluorescein) and the quencher was BHQ1 (Black Hole Quencher 1). The underlined portion hybridizes with a region of the Z3276 gene sequence corresponding to a portion of the LB primer.

In this example, the LAMP reaction to detect the target is carried out in a 25 μl volume (total) containing the following components: Primer Mix C, 6 mM MgSO₄, 1.4 mM of each dNTP, 1× isothermal amplification reaction buffer (where a 10× solution is composed of 200 mM of Tris-HCl pH 8.8, 100 mM of (NH₄)₂SO₄, 100 mM of KCl, 20 mM MgSO₄, and 1% Tween-20), 8 Units of Bst Wild-Type Large Fragment polymerase (New England BioLabs), and at least 100 copies of target DNA. The reaction mixture is made by combining the reagents as indicated in Tables 4 and 5.

The LAMP amplification is carried out using the Bio-Rad CFX96 Touch Real-Time PCR Detection System (Hercules, California, USA). The reaction mixture is heated at 60° C. for 20 minutes. Negative and positive controls are included in each run.

Detection:

Generation of the reaction product was detected using the Bio-Rad CFX96 Touch Real-Time PCR Detection System. 25 μl reactions were aliquoted into single wells of a standard 96-well microtiter plate and placed as per machine manufacturer's instructions into the instrument and into the path of a fluorescent beam using the FAM channel to excite the fluorescent moiety of the molecular beacon probe. A signal in the emission wavelength of the fluorescent moiety was detected. A negative control containing no target DNA template (with the same volume substituted with nuclease-free water in the final master mix) was used.

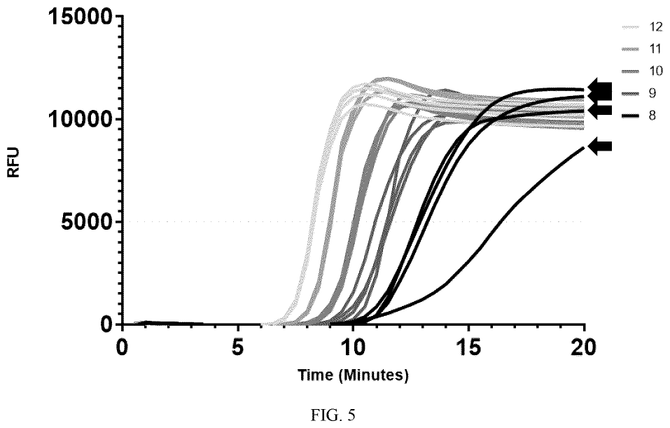

FIG. 5 shows amplification curves demonstrating transferable utility from one primer set to another. The primer set used was designed for another DNA target sequence, and uses the concentrations stated in Primer Mix C, Table 3, in a multiplex format. Samples #8-12 are in a multiplex assay format. Sample #12 contains 10\hat{ }5 copies of the target. Sample #11 contains 10\hat{ }4 copies of the target. Sample #10 contains 10\hat{ }3 copies of the target. Sample #9 contains 10\hat{ }2 copies of the target. Sample #8 contains 10\hat{ }1 copies of the target. The four replicates for Sample #8 are denoted with the darkest black arrows and corresponding darkest black lines.

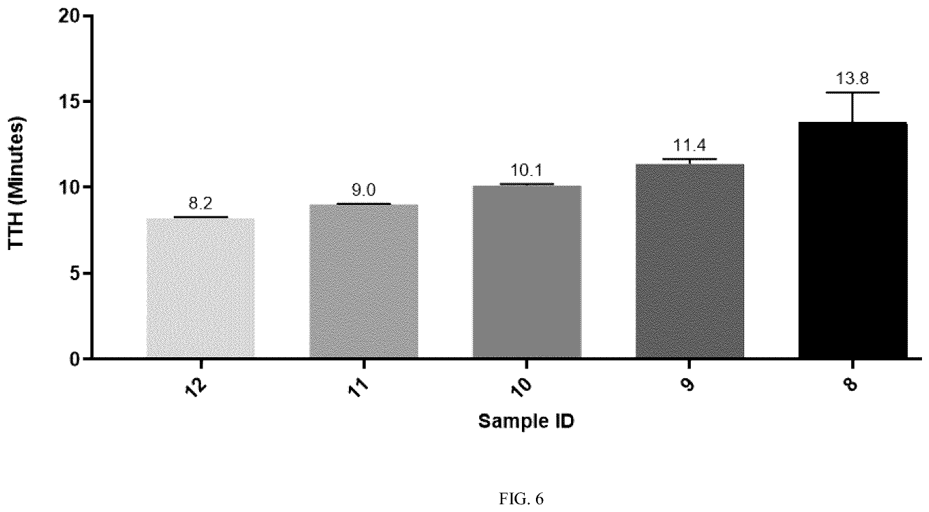

FIG. 6: TTH was defined as the time elapsed until the amplification curve reached a minimum threshold height (5000 RFU), as shown in FIG. 5.

### Example 4

In this Example, the following primers were used:

F3 primer
(SEQ ID NO: 1)
5′-GAACGTGTCGCGGAAGTC-3′
B3 primer
(SEQ ID NO: 2)
5′-CGGCAATAGCGTCACCTT-3′
FIP primer
(SEQ ID NO: 3)
5′-GCGCGGCATCCGCATCAATATCTGGATGGTATGCCCGG-3′
BIP primer
(SEQ ID NO: 4)
5′-GCGAACGGCGAAGCGTACTGTCGCACCGTCAAAGGAAC-3′
LoopF primer
(SEQ ID NO: 5)
5′-TCAAATCGGCATCAATACTCATCTG-3′
LoopB primer
(SEQ ID NO: 6)
5′-AAAGGGAAAGCCAGCTTTACG-3′

In this example, the target was a portion of a nucleic acid encoding the Invasion A protein of Salmonella typhimurium. The target sequence was:

(SEQ ID NO: 7)
5′GAACGTGTCGCGGAAGTCGCGGCCCGATTTTCTCTGGATGGTATGCCC
GGTAAACAGATGAGTATTGATGCCGATTTGAAGGCCGGTATTATTGATGC
GGATGCCGCGCGCGAACGGCGAAGCGTACTGGAAAGGGAAAGCCAGCTTT
ACGGTTCCTTTGACGGTGCGATGAAGTTTATCAAAGGTGACGCTATTGCC
G-3′

The Molecular Beacon probe used in this example has the sequence:

(SEQ ID NO: 8)
5′-CGTAAAGGGAAAGCCAGCTTTACG-3′

The underlined portion of the molecular beacon sequence hybridizes with a region of the Invasion A gene sequence corresponding to a portion of the LB primer. A fluorophore and quencher were attached to the oligonucleotide of the molecular beacon, the fluorophore was FAM (6-carboxyfluorescein) and the quencher was BHQ1 (Black Hole Quencher 1).

In this example, the LAMP reaction to detect the target is carried out in a 25 μl volume (total) containing the following components: Primer Mix A, Primer Mix B, or Primer Mix B shown in Table 2, except the concentration of the LF and LB primers were included at an equal ratio of 1:1 (0.6 μM) each, 6 mM MgSO₄, 1.4 mM of each dNTP, 1× isothermal amplification reaction buffer (where a 10× solution is composed of 200 mM of Tris-HCl pH 8.8, 100 mM of (NH₄)₂SO₄, 100 mM of KCl, 20 mM MgSO₄, and 1% Tween-20), 8 Units of Bst Wild-Type Large Fragment polymerase (New England BioLabs), and at least 100 copies of target DNA. The reaction mixture is made by combining the reagents as indicated in Tables 4 and 5.

The LAMP amplification is carried out using the Bio-Rad CFX96 Touch Real-Time PCR Detection System (Hercules, California, USA). The reaction mixture is heated at 60° C. for 20 minutes. Negative and positive controls are included in each run.

Detection

Generation of the reaction product was detected using the Bio-Rad CFX96 Touch Real-Time PCR Detection System. 25 μl reactions were aliquoted into single wells of a standard 96-well microtiter plate and placed as per machine manufacturer's instructions into the instrument and into the path of a fluorescent beam using the FAM channel to excite the fluorescent moiety of the molecular beacon probe. A signal in the emission wavelength of the fluorescent moiety was detected. A negative control containing no target DNA template (with the same volume substituted with nuclease-free water in the final master mix) was used.

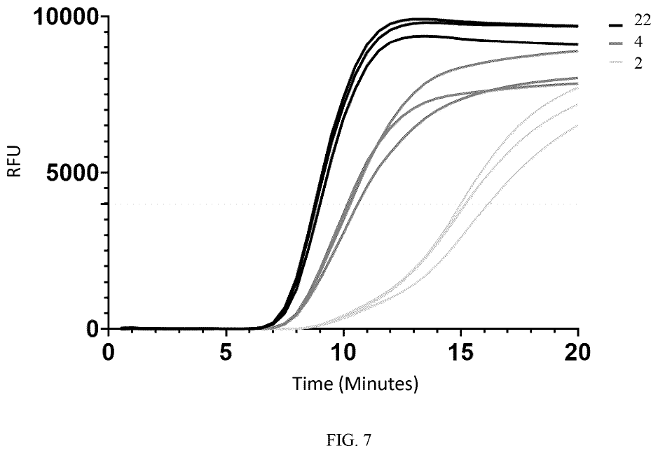

FIG. 7: Direct comparison of LAMP assay performance differences in sensitivity. Samples #2, 4, and 22 contain 10\hat{ }2 copies of target. Sample #2 used Primer Mix A shown in Table 1. Sample #4 used Primer Mix B shown in Table 2, except the concentration of the LF and LB primers were evaluated at an equal ratio of 1:1 (0.6 μM) each. Sample #22 used Primer Mix B shown in Table 2.

### Example 5

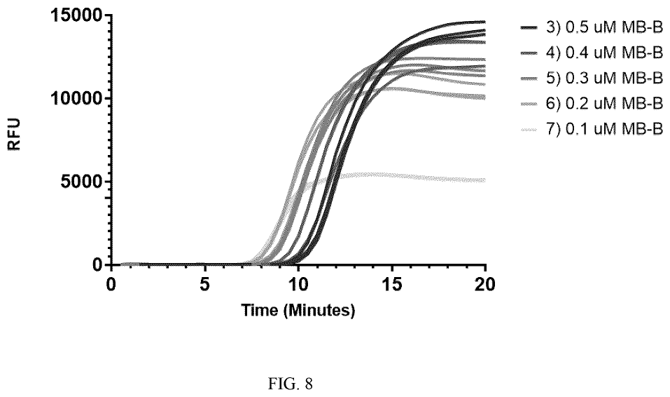

In this example, the primer mix used had the concentrations stated in Primer Mix B, Table 2, except that the LF and LB were equal in concentration (0.6 μM) and the concentration of molecular beacon (MB-B) was varied from 0.1 micromolar to 0.5 micromolar. Results are shown in FIG. 8 and show that for a target assay time under 10 minutes, less than 0.3 μM of molecular beacon is desirable.

### Example 6

In this Example, the following primers were used:

F3 primer
(SEQ ID NO: 1)
5′-GAACGTGTCGCGGAAGTC-3′
B3 primer
(SEQ ID NO: 2)
5′-CGGCAATAGCGTCACCTT-3′
FIP primer
(SEQ ID NO: 3)
5′-GCGCGGCATCCGCATCAATATCTGGATGGTATGCCCGG-3′
BIP primer
(SEQ ID NO: 4)
5′-GCGAACGGCGAAGCGTACTGTCGCACCGTCAAAGGAAC-3′
LoopF primer
(SEQ ID NO: 5)
5′-TCAAATCGGCATCAATACTCATCTG-3′
LoopB primer
(SEQ ID NO: 6)
5′-AAAGGGAAAGCCAGCTTTACG-3′

In this example, the target was a portion of a nucleic acid encoding the Invasion A protein of Salmonella typhimurium. The target sequence was:

(SEQ ID NO: 7)
5′GAACGTGTCGCGGAAGTCGCGGCCCGATTTTCTCTGGATGGTATGCCC
GGTAAACAGATGAGTATTGATGCCGATTTGAAGGCCGGTATTATTGATGC
GGATGCCGCGCGCGAACGGCGAAGCGTACTGGAAAGGGAAAGCCAGCTTT
ACGGTTCCTTTGACGGTGCGATGAAGTTTATCAAAGGTGACGCTATTGCC
G-3′

The Molecular Beacon probe used in this example has the sequence:

(SEQ ID NO: 8)
5′-CGTAAAGGGAAAGCCAGCTTTACG-3′

The underlined portion of the molecular beacon sequence hybridizes with a region of the Invasion A gene sequence corresponding to a portion of the LB primer. A fluorophore and quencher were attached to the oligonucleotide of the molecular beacon, the fluorophore was FAM (6-carboxyfluorescein) and the quencher was BHQ1 (Black Hole Quencher 1).

In this example, the LAMP reaction to detect the target is carried out in a 25 μl volume (total) containing the following components: Primer Mix A, Table 1, except for variation in FIP and BIP ratios, FIP:BIP, sample IDs 1-32, which were varied as shown in Table 6, 6 mM MgSO₄, 1.4 mM of each dNTP, 1× isothermal amplification reaction buffer (where a 10× solution is composed of 200 mM of Tris-HCl pH 8.8, 100 mM of (NH₄)₂SO₄, 100 mM of KCl, 20 mM MgSO₄, and 1% Tween-20), 8 Units of Bst Wild-Type Large Fragment polymerase (New England BioLabs), and at least 100 copies of target DNA. The reaction mixture is made by combining the reagents as indicated in Tables 4 and 5.

The LAMP amplification is carried out using the Bio-Rad CFX96 Touch Real-Time PCR Detection System (Hercules, California, USA). The reaction mixture is heated at 60° C. for 20 minutes. Negative and positive controls are included in each run.

Detection

Generation of the reaction product was detected using the Bio-Rad CFX96 Touch Real-Time PCR Detection System. 25 μl reactions were aliquoted into single wells of a standard 96-well microtiter plate and placed as per machine manufacturer's instructions into the instrument and into the path of a fluorescent beam using the FAM channel to excite the fluorescent moiety of the molecular beacon probe. A signal in the emission wavelength of the fluorescent moiety was detected. A negative control containing no target DNA template (with the same volume substituted with nuclease-free water in the final master mix) was used.

TABLE 6

| Ratio of | FIP | BIP | |
|---|---|---|---|
| FIP:BIP | (Final Conc. μM) | (Final Conc. μM) | Sample ID |
| 1.00 | 2.0 | 2.0 | 1 |
| 1.11 | 2.0 | 1.8 | 2 |
| 1.25 | 2.0 | 1.6 | 3 |
| 1.43 | 2.0 | 1.4 | 4 |
| 2.00 | 2.0 | 1.0 | 5 |
| 3.33 | 2.0 | 0.6 | 6 |
| 1.00 | 1.8 | 1.8 | 7 |
| 1.13 | 1.8 | 1.6 | 8 |
| 1.29 | 1.8 | 1.4 | 9 |
| 1.80 | 1.8 | 1.0 | 10 |
| 3.00 | 1.8 | 0.6 | 11 |
| 0.89 | 1.6 | 1.8 | 12 |
| 1.00 | 1.6 | 1.6 | 13 |
| — | — | — | 14 |
| 1.14 | 1.6 | 1.4 | 15 |
| 1.60 | 1.6 | 1.0 | 16 |
| 2.67 | 1.6 | 0.6 | 17 |
| 0.78 | 1.4 | 1.8 | 18 |
| 0.88 | 1.4 | 1.6 | 19 |
| 1.00 | 1.4 | 1.4 | 20 |
| 1.40 | 1.4 | 1.0 | 21 |
| 2.33 | 1.4 | 0.6 | 22 |
| 0.56 | 1.0 | 1.8 | 23 |
| 0.63 | 1.0 | 1.6 | 24 |
| 0.71 | 1.0 | 1.4 | 25 |
| 1.00 | 1.0 | 1.0 | 26 |
| 1.67 | 1.0 | 0.6 | 27 |
| 0.33 | 0.6 | 1.8 | 28 |
| 0.38 | 0.6 | 1.6 | 29 |
| 0.43 | 0.6 | 1.4 | 30 |
| 0.60 | 0.6 | 1.0 | 31 |
| 1.00 | 0.6 | 0.6 | 32 |

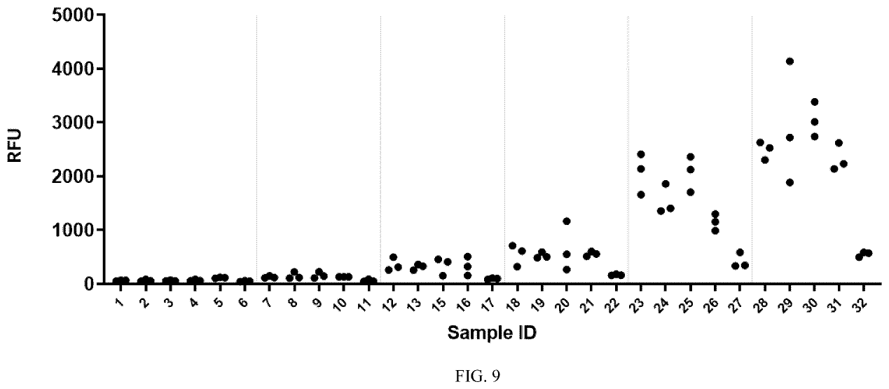

FIG. 9 is a graph showing results of LAMP assays using Primer Mix A, Table 1, except for variation in FIP and BIP ratios, FIP:BIP, sample IDs 1-32, which were varied as shown in Table 6. The graph of FIG. 9 shows the resulting end-point RFU after 10 minutes of assay time, where the highest RFU is most desirable at this given time point.

### Example 7

In this Example, the following primers were used:

F3 primer
(SEQ ID NO: 1)
5′-GAACGTGTCGCGGAAGTC-3′
B3 primer
(SEQ ID NO: 2)
5′-CGGCAATAGCGTCACCTT-3′
FIP primer
(SEQ ID NO: 3)
5′-GCGCGGCATCCGCATCAATATCTGGATGGTATGCCCGG-3′
BIP primer
(SEQ ID NO: 4)
5′-GCGAACGGCGAAGCGTACTGTCGCACCGTCAAAGGAAC-3′
LoopF primer
(SEQ ID NO: 5)
5′-TCAAATCGGCATCAATACTCATCTG-3′
LoopB primer
(SEQ ID NO: 6)
5′-AAAGGGAAAGCCAGCTTTACG-3′

In this example, the target was a portion of a nucleic acid encoding the Invasion A protein of Salmonella typhimurium. The target sequence was:

(SEQ ID NO: 7)
5′GAACGTGTCGCGGAAGTCGCGGCCCGATTTTCTCTGGATGGTATGCCC
GGTAAACAGATGAGTATTGATGCCGATTTGAAGGCCGGTATTATTGATGC
GGATGCCGCGCGCGAACGGCGAAGCGTACTGGAAAGGGAAAGCCAGCTTT
ACGGTTCCTTTGACGGTGCGATGAAGTTTATCAAAGGTGACGCTATTGCC
G-3′

The Molecular Beacon probe used in this example has the sequence:

(SEQ ID NO: 8)
5′-CGTAAAGGGAAAGCCAGCTTTACG-3′

The underlined portion of the molecular beacon sequence hybridizes with a region of the Invasion A gene sequence corresponding to a portion of the LB primer. A fluorophore and quencher were attached to the oligonucleotide of the molecular beacon, the fluorophore was FAM (6-carboxyfluorescein) and the quencher was BHQ1 (Black Hole Quencher 1).

In this example, the LAMP reaction to detect the target is carried out in a 25 μl volume (total) containing the following components: Primer Mix A, Table 1, except for variation in FIP and BIP ratios, FIP:BIP, sample IDs 1-32, which were varied as shown in Table 7, 6 mM MgSO₄, 1.4 mM of each dNTP, 1× isothermal amplification reaction buffer (where a 10× solution is composed of 200 mM of Tris-HCl pH 8.8, 100 mM of (NH₄)₂SO₄, 100 mM of KCl, 20 mM MgSO₄, and 1% Tween-20), 8 Units of Bst Wild-Type Large Fragment polymerase (New England BioLabs), and at least 100 copies of target DNA. The reaction mixture is made by combining the reagents as indicated in Tables 4 and 5.

The LAMP amplification is carried out using the Bio-Rad CFX96 Touch Real-Time PCR Detection System (Hercules, California, USA). The reaction mixture is heated at 60° C. for 20 minutes. Negative and positive controls are included in each run.

Detection

Generation of the reaction product was detected using the Bio-Rad CFX96 Touch Real-Time PCR Detection System. 25 μl reactions were aliquoted into single wells of a standard 96-well microtiter plate and placed as per machine manufacturer's instructions into the instrument and into the path of a fluorescent beam using the FAM channel to excite the fluorescent moiety of the molecular beacon probe. A signal in the emission wavelength of the fluorescent moiety was detected. A negative control containing no target DNA template (with the same volume substituted with nuclease-free water in the final master mix) was used.

TABLE 7

| Ratio of | FIP | BIP | |
|---|---|---|---|
| FIP:BIP | (Final Conc. μM) | (Final Conc. μM) | Sample ID |
| — | — | — | 1 |
| — | — | — | 2 |
| 0.38 | 0.6 | 1.6 | 3 |
| 0.40 | 0.6 | 1.5 | 4 |
| 0.43 | 0.6 | 1.4 | 5 |
| 0.46 | 0.6 | 1.3 | 6 |
| 0.50 | 0.6 | 1.2 | 7 |
| 0.55 | 0.6 | 1.1 | 8 |
| 0.31 | 0.5 | 1.6 | 9 |
| 0.33 | 0.5 | 1.5 | 10 |
| 0.36 | 0.5 | 1.4 | 11 |
| 0.38 | 0.5 | 1.3 | 12 |
| 0.42 | 0.5 | 1.2 | 13 |
| 0.45 | 0.5 | 1.1 | 14 |
| 0.25 | 0.4 | 1.6 | 15 |
| 0.27 | 0.4 | 1.5 | 16 |
| 0.29 | 0.4 | 1.4 | 17 |
| 0.31 | 0.4 | 1.3 | 18 |
| 0.33 | 0.4 | 1.2 | 19 |
| 0.36 | 0.4 | 1.1 | 20 |
| 0.19 | 0.3 | 1.6 | 21 |
| 0.20 | 0.3 | 1.5 | 22 |
| 0.21 | 0.3 | 1.4 | 23 |
| 0.23 | 0.3 | 1.3 | 24 |
| 0.25 | 0.3 | 1.2 | 25 |
| 0.27 | 0.3 | 1.1 | 26 |
| 0.13 | 0.2 | 1.6 | 27 |
| 0.13 | 0.2 | 1.5 | 28 |
| 0.14 | 0.2 | 1.4 | 29 |
| 0.15 | 0.2 | 1.3 | 30 |
| 0.17 | 0.2 | 1.2 | 31 |
| 0.18 | 0.2 | 1.1 | 32 |

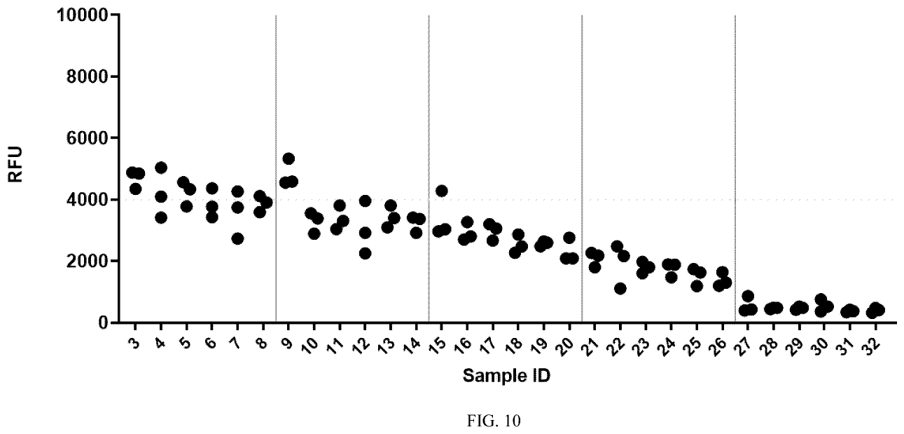

FIG. 10 is a graph showing results of LAMP assays using Primer Mix A, Table 1, except for variation in FIP and BIP ratios, FIP:BIP, sample IDs 1-32, which were varied as shown in Table 7. The graph of FIG. 10 shows the resulting end-point RFU after 10 minutes of assay time, where the highest RFU is most desirable at this given time point.

### Example 8

In this Example, the following primers were used:

F3 primer
(SEQ ID NO: 1)
5′-GAACGTGTCGCGGAAGTC-3′
B3 primer
(SEQ ID NO: 2)
5′-CGGCAATAGCGTCACCTT-3′
FIP primer
(SEQ ID NO: 3)
5′-GCGCGGCATCCGCATCAATATCTGGATGGTATGCCCGG-3′
BIP primer
(SEQ ID NO: 4)
5′-GCGAACGGCGAAGCGTACTGTCGCACCGTCAAAGGAAC-3′
LoopF primer
(SEQ ID NO: 5)
5′-TCAAATCGGCATCAATACTCATCTG-3′
LoopB primer
(SEQ ID NO: 6)
5′-AAAGGGAAAGCCAGCTTTACG-3′

In this example, the target was a portion of a nucleic acid encoding the Invasion A protein of Salmonella typhimurium. The target sequence was:

(SEQ ID NO: 7)
5′GAACGTGTCGCGGAAGTCGCGGCCCGATTTTCTCTGGATGGTATGCCC
GGTAAACAGATGAGTATTGATGCCGATTTGAAGGCCGGTATTATTGATGC
GGATGCCGCGCGCGAACGGCGAAGCGTACTGGAAAGGGAAAGCCAGCTTT
ACGGTTCCTTTGACGGTGCGATGAAGTTTATCAAAGGTGACGCTATTGCC
G-3′

The Molecular Beacon probe used in this example has the sequence:

(SEQ ID NO: 8)
5′-CGTAAAGGGAAAGCCAGCTTTACG-3′

The underlined portion of the molecular beacon sequence hybridizes with a region of the Invasion A gene sequence corresponding to a portion of the LB primer. A fluorophore and quencher were attached to the oligonucleotide of the molecular beacon, the fluorophore was FAM (6-carboxyfluorescein) and the quencher was BHQ1 (Black Hole Quencher 1).

In this example, the LAMP reaction to detect the target is carried out in a 25 μl volume (total) containing the following components: Primer Mix B, Table 2, except for variation in LF and LB ratios, LF:LB, sample IDs 1-32, which were varied as shown in Table 8, 6 mM MgSO₄, 1.4 mM of each dNTP, 1× isothermal amplification reaction buffer (where a 10× solution is composed of 200 mM of Tris-HCl pH 8.8, 100 mM of (NH₄)₂SO₄, 100 mM of KCl, 20 mM MgSO₄, and 1% Tween-20), 8 Units of Bst Wild-Type Large Fragment polymerase (New England BioLabs), and at least 100 copies of target DNA. The reaction mixture is made by combining the reagents as indicated in Tables 4 and 5.

The LAMP amplification is carried out using the Bio-Rad CFX96 Touch Real-Time PCR Detection System (Hercules, California, USA). The reaction mixture is heated at 60° C. for 20 minutes. Negative and positive controls are included in each run.

Detection

Generation of the reaction product was detected using the Bio-Rad CFX96 Touch Real-Time PCR Detection System. 25 μl reactions were aliquoted into single wells of a standard 96-well microtiter plate and placed as per machine manufacturer's instructions into the instrument and into the path of a fluorescent beam using the FAM channel to excite the fluorescent moiety of the molecular beacon probe. A signal in the emission wavelength of the fluorescent moiety was detected. A negative control containing no target DNA template (with the same volume substituted with nuclease-free water in the final master mix) was used.

TABLE 8

| Ratio of | LF | LB | |
|---|---|---|---|
| LF:LB | (Final Conc. μM) | (Final Conc. (μM) | Sample ID |
| | — | — | 1 |
| | — | — | 2 |
| 1.50 | 0.6 | 0.4 | 3 |
| 1.00 | 0.6 | 0.6 | 4 |
| 0.75 | 0.6 | 0.8 | 5 |
| 0.60 | 0.6 | 1.0 | 6 |
| 0.50 | 0.6 | 1.2 | 7 |
| 0.43 | 0.6 | 1.4 | 8 |
| 2.00 | 0.8 | 0.4 | 9 |
| 1.33 | 0.8 | 0.6 | 10 |
| 1.00 | 0.8 | 0.8 | 11 |
| 0.80 | 0.8 | 1.0 | 12 |
| 0.67 | 0.8 | 1.2 | 13 |
| 0.57 | 0.8 | 1.4 | 14 |
| 2.50 | 1.0 | 0.4 | 15 |
| 1.67 | 1.0 | 0.6 | 16 |
| 1.25 | 1.0 | 0.8 | 17 |
| 1.00 | 1.0 | 1.0 | 18 |
| 0.83 | 1.0 | 1.2 | 19 |
| 0.71 | 1.0 | 1.4 | 20 |
| 3.00 | 1.2 | 0.4 | 21 |
| 2.00 | 1.2 | 0.6 | 22 |
| 1.50 | 1.2 | 0.8 | 23 |
| 1.20 | 1.2 | 1.0 | 24 |
| 1.00 | 1.2 | 1.2 | 25 |
| 0.86 | 1.2 | 1.4 | 26 |
| 3.50 | 1.4 | 0.4 | 27 |
| 2.33 | 1.4 | 0.6 | 28 |
| 1.75 | 1.4 | 0.8 | 29 |
| 1.40 | 1.4 | 1.0 | 30 |
| 1.17 | 1.4 | 1.2 | 31 |
| 1.00 | 1.4 | 1.4 | 32 |

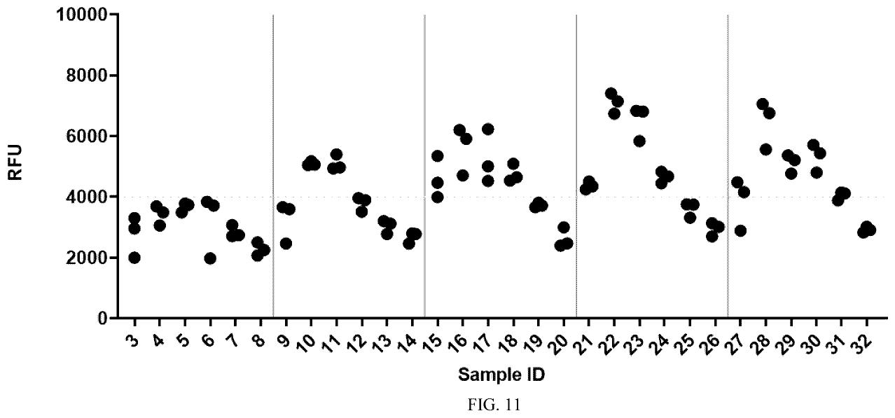

FIG. 11 is a graph showing results of LAMP assays using Primer Mix B, Table 2, except for variation in LF and LB ratios, LF:LB, sample IDs 1-32, which were varied as shown in Table 8. The graph of FIG. 11 shows the resulting end-point RFU after 10 minutes of assay time, where the highest RFU is most desirable at this given time point.

### Example 9

In this Example, the following primers were used:

F3 primer
(SEQ ID NO: 1)
5′-GAACGTGTCGCGGAAGTC-3′
B3 primer
(SEQ ID NO: 2)
5′-CGGCAATAGCGTCACCTT-3′
FIP primer
(SEQ ID NO: 3)
5′-GCGCGGCATCCGCATCAATATCTGGATGGTATGCCCGG-3′
BIP primer
(SEQ ID NO: 4)
5′-GCGAACGGCGAAGCGTACTGTCGCACCGTCAAAGGAAC-3′
LoopF primer
(SEQ ID NO: 5)
5′-TCAAATCGGCATCAATACTCATCTG-3′
LoopB primer
(SEQ ID NO: 6)
5′-AAAGGGAAAGCCAGCTTTACG-3′

In this example, the target was a portion of a nucleic acid encoding the Invasion A protein of Salmonella typhimurium. The target sequence was:

(SEQ ID NO: 7)
5′GAACGTGTCGCGGAAGTCGCGGCCCGATTTTCTCTGGATGGTATGCCC
GGTAAACAGATGAGTATTGATGCCGATTTGAAGGCCGGTATTATTGATGC
GGATGCCGCGCGCGAACGGCGAAGCGTACTGGAAAGGGAAAGCCAGCTTT
ACGGTTCCTTTGACGGTGCGATGAAGTTTATCAAAGGTGACGCTATTGCC
G-3′

The Molecular Beacon probe used in this example has the sequence:

(SEQ ID NO: 8)
5′-CGTAAAGGGAAAGCCAGCTTTACG-3′

The underlined portion of the molecular beacon sequence hybridizes with a region of the Invasion A gene sequence corresponding to a portion of the LB primer. A fluorophore and quencher were attached to the oligonucleotide of the molecular beacon, the fluorophore was FAM (6-carboxyfluorescein) and the quencher was BHQ1 (Black Hole Quencher 1).

In this example, the LAMP reaction to detect the target is carried out in a 25 μl volume (total) containing the following components: Primer Mix B, Table 2, except for variation in F3 and B3 ratios, F3:B3, sample IDs 1-32, which were varied as shown in Table 9, 6 mM MgSO₄, 1.4 mM of each dNTP, 1× isothermal amplification reaction buffer (where a 10× solution is composed of 200 mM of Tris-HCl pH 8.8, 100 mM of (NH₄)₂SO₄, 100 mM of KCl, 20 mM MgSO₄, and 1% Tween-20), 8 Units of Bst Wild-Type Large Fragment polymerase (New England BioLabs), and at least 100 copies of target DNA. The reaction mixture is made by combining the reagents as indicated in Tables 4 and 5.

The LAMP amplification is carried out using the Bio-Rad CFX96 Touch Real-Time PCR Detection System (Hercules, California, USA). The reaction mixture is heated at 60° C. for 20 minutes. Negative and positive controls are included in each run.

Detection

Generation of the reaction product was detected using the Bio-Rad CFX96 Touch Real-Time PCR Detection System. 25 μl reactions were aliquoted into single wells of a standard 96-well microtiter plate and placed as per machine manufacturer's instructions into the instrument and into the path of a fluorescent beam using the FAM channel to excite the fluorescent moiety of the molecular beacon probe. A signal in the emission wavelength of the fluorescent moiety was detected. A negative control containing no target DNA template (with the same volume substituted with nuclease-free water in the final master mix) was used.

TABLE 9

| Ratio of | F3 | B3 | |
|---|---|---|---|
| F3:B3 | (Final Conc. (μM) | (Final Conc. (μM) | Sample ID |
| — | — | — | 1 |
| — | — | — | 2 |
| 1.00 | 0.2 | 0.2 | 3 |
| 0.50 | 0.2 | 0.4 | 4 |
| 0.33 | 0.2 | 0.6 | 5 |
| 0.25 | 0.2 | 0.8 | 6 |
| 0.20 | 0.2 | 1.0 | 7 |
| 0.17 | 0.2 | 1.2 | 8 |
| 2.00 | 0.4 | 0.2 | 9 |
| 1.00 | 0.4 | 0.4 | 10 |
| 0.67 | 0.4 | 0.6 | 11 |
| 0.50 | 0.4 | 0.8 | 12 |
| 0.40 | 0.4 | 1.0 | 13 |
| 0.33 | 0.4 | 1.2 | 14 |
| 3.00 | 0.6 | 0.2 | 15 |
| 1.50 | 0.6 | 0.4 | 16 |
| 1.00 | 0.6 | 0.6 | 17 |
| 0.75 | 0.6 | 0.8 | 18 |
| 0.60 | 0.6 | 1.0 | 19 |
| 0.50 | 0.6 | 1.2 | 20 |
| 4.00 | 0.8 | 0.2 | 21 |
| 2.00 | 0.8 | 0.4 | 22 |
| 1.33 | 0.8 | 0.6 | 23 |
| 1.00 | 0.8 | 0.8 | 24 |
| 0.80 | 0.8 | 1.0 | 25 |
| 0.67 | 0.8 | 1.2 | 26 |
| 5.00 | 1.0 | 0.2 | 27 |
| 2.50 | 1.0 | 0.4 | 28 |
| 1.67 | 1.0 | 0.6 | 29 |
| 1.25 | 1.0 | 0.8 | 30 |
| 1.00 | 1.0 | 1.0 | 31 |
| 0.83 | 1.0 | 1.2 | 32 |

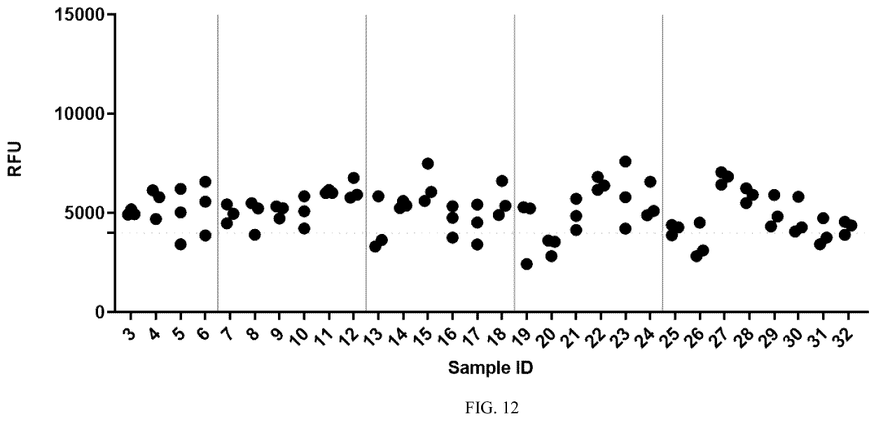

FIG. 12 is a graph showing results of LAMP assays using Primer Mix B, Table 2, except for variation in F3 and B3 ratios, F3:B3, sample IDs 1-32, which were varied as shown in Table 9. The graph of FIG. 12 shows the resulting end-point RFU after 10 minutes of assay time, where the highest RFU is most desirable at this given time point.

Item List

Item 1. A method for detection of a target nucleic acid in a sample, comprising: providing a reaction mixture comprising a LAMP assay primer set specific for the target nucleic acid, magnesium, dNTPs, a reaction buffer, a DNA polymerase, and a sample to be tested for presence of the target nucleic acid, wherein the LAMP assay primer set comprises a forward inner primer (FIP), a backward inner primer (BIP), a forward outer primer (F3) and a backward outer primer (B3), wherein the FIP and BIP are present in a non-equal ratio such that molar concentration of BIP is highly skewed relative to molar concentration of FIP; incubating the reaction mixture under amplification reaction conditions to produce a reaction product comprising amplified target nucleic acids, wherein the reaction product comprises a forward strand and a complementary backward strand, the forward strand comprising the FIP, the backward strand comprising the BIP; and detecting the amplified target nucleic acids.

Item 2. The method of item 1, wherein BIP has a molar concentration at least 12% higher than the molar concentration of FIP and no more than 300% higher than the molar concentration of FIP, or FIP has a molar concentration at least 12% higher than the molar concentration of BIP and no more than 300% higher than the molar concentration of BIP.

Item 3. The method of item 1 or item 2, wherein BIP has a molar concentration at least 50% higher than the molar concentration of FIP and no more than 300% higher than the molar concentration of FIP, or FIP has a molar concentration at least 50% higher than the molar concentration of BIP and no more than 300% higher than the molar concentration of BIP.

Item 4. The method of any one of items 1, 2, or 3, wherein the F3 and B3 are present in an equal ratio.

Item 5. The method of any one of items 1, 2, or 3, wherein the F3 and B3 are present in a non-equal ratio such that when FIP is present in a greater molar concentration than BIP, F3 is present in a greater molar concentration than B3, and wherein when BIP is present in a greater molar concentration than FIP, B3 is present in a greater molar concentration than F3.

Item 6. The method of item 5, wherein when BIP has a molar concentration at least 12% higher than the molar concentration of FIP and no more than 300% higher than the molar concentration of FIP, B3 has a molar concentration higher than the molar concentration of F3 and no more than 1000% higher than the molar concentration of F3.

Item 7. The method of item 5, wherein when BIP has a molar concentration at least 50% higher than the molar concentration of FIP and no more than 300% higher than the molar concentration of FIP, B3 has a molar concentration higher than the molar concentration of F3 and no more than 1000% higher than the molar concentration of F3.

Item 8. The method of item 5, wherein when FIP has a molar concentration at least 12% higher than the molar concentration of BIP and no more than 300% higher than the molar concentration of BIP, F3 has a molar concentration higher than the molar concentration of B3 and no more than 1000% higher than the molar concentration of B3.

Item 9. The method of item 5, wherein when FIP has a molar concentration at least 50% higher than the molar concentration of BIP and no more than 300% higher than the molar concentration of BIP, F3 has a molar concentration higher than the molar concentration of B3 and no more than 1000% higher than the molar concentration of B3.

Item 10. The method of any one of items 1 to 9, wherein the reaction mixture further comprises a Loop F (LF) primer, a Loop B (LB) primer, or both a Loop F (LF) primer and a Loop B (LB) primer, wherein the LF and LB are present in a non-equal ratio when both are present, such that molar concentration of LF is highly skewed relative to LB.

Item 11. The method of item 10, wherein when BIP has a molar concentration at least 12% higher than the molar concentration of FIP and no more than 300% higher than the molar concentration of FIP, LB is present and no LF is present, or LB has a molar concentration at least 12% higher than the molar concentration of LF and no more than 500% higher than the molar concentration of LF when both LB and LF are present.

Item 12. The method of item 10, wherein when BIP has a molar concentration at least 50% higher than the molar concentration of FIP and no more than 300% higher than the molar concentration of FIP, LB is present and no LF is present, or LB has a molar concentration at least 50% higher than the molar concentration of LF and no more than 500% higher than the molar concentration of LF when both LB and LF are present.

Item 13. The method of item 10, wherein when FIP has a molar concentration at least 12% higher than the molar concentration of BIP and no more than 300% higher than the molar concentration of BIP, LF is present and no LB is present, or LF has a molar concentration at least 12% higher than the molar concentration of LB and no more than 500% higher than the molar concentration of LB when both LB and LF are present.

Item 14. The method of item 10, wherein when FIP has a molar concentration at least 50% higher than the molar concentration of BIP and no more than 300% higher than the molar concentration of BIP, LF is present and no LB is present, or LF has a molar concentration at least 50% higher than the molar concentration of LB and no more than 500% higher than the molar concentration of LB when both LB and LF are present.

Item 15. The method of any one of items 1 to 14, wherein detecting the amplified target nucleic acids, comprises detecting one or more of: pH, turbidity, an electrophoresis pattern, and a detectable label.

Item 16. The method of item 15, wherein detecting the amplified target nucleic acids comprises detecting a detectable label, and wherein the detectable label comprises one or more of: a fluorescent label, a bioluminescent label, a chemiluminescent label, a chromophore, a magnetic label, an enzyme, a substrate, and a radioisotope.

Item 17. The method of item 15, wherein the detectable label comprises a fluorescence resonance energy transfer (FRET) fluorescent label.

Item 18. The method of item 15, wherein the detectable label comprises a fluorescent label comprising a fluorescent intercalating dye.

Item 19. The method of any one of items 15 to 18, wherein the detectable label is present in one or more primers of the LAMP assay primer set.

Item 20. The method of any one of items 16 to 19, wherein the detectable label is a fluorescent label present in one or more primers of the LAMP assay primer set, incorporated into the amplified target nucleic acids.

Item 21. The method of any one of items 15 to 20, wherein the detectable label is a fluorescent label present in a probe, and wherein the probe is a sequence-specific binding partner for a specified sequence present in the amplified target nucleic acids.

Item 22. The method of item 21, wherein the probe is a sequence-specific binding partner for a specified sequence present in the forward strand of the amplified target nucleic acids when FIP has a molar concentration at least 12% higher than the molar concentration of BIP and no more than 300% higher than the molar concentration of BIP.

Item 23. The method of item 21, wherein the probe is a sequence-specific binding partner for a specified sequence present in the forward strand of the amplified target nucleic acids when FIP has a molar concentration at least 50% higher than the molar concentration of BIP and no more than 300% higher than the molar concentration of BIP.

Item 24. The method of item 21, wherein the probe is a sequence-specific binding partner for a specified sequence present in the backward strand of the amplified target nucleic acids when BIP has a molar concentration at least 12% higher than the molar concentration of FIP and no more than 300% higher than the molar concentration of FIP.

Item 25. The method of item 21, wherein the probe is a sequence-specific binding partner for a specified sequence present in the backward strand of the amplified target nucleic acids when BIP has a molar concentration at least 50% higher than the molar concentration of FIP and no more than 300% higher than the molar concentration of FIP.

Item 26. The method of any one of items 21 to 25, wherein the probe is a fluorescence resonance energy transfer (FRET)-labeled oligonucleotide probe, wherein the FRET label comprises a quencher and a fluorescent moiety such that the quencher quenches a fluorescent signal of the fluorescent moiety when the oligonucleotide probe is in stem-loop configuration and not hybridized to the specified sequence, and wherein the fluorescent signal of the fluorescent moiety is detectable when the fluorescently-labeled oligonucleotide probe is specifically hybridized to the specified sequence.

Item 27. The method of any one of items 1 to 26, wherein the reaction mixture further comprises a reverse transcriptase.

Item 28. A method for detection of a target nucleic acid in a sample, comprising: providing a reaction mixture comprising: a LAMP assay primer set specific for the target nucleic acid, magnesium, dNTPs, a reaction buffer, a DNA polymerase, and a sample to be tested for presence of the target nucleic acid, wherein the LAMP assay primer set comprises 1) a forward inner primer (FIP), 2) a backward inner primer (BIP), 3) a forward outer primer (F3), 4) a backward outer primer (B3), and 5) a Loop F (LF) primer or a Loop B (LB) primer, or both a Loop F (LF) primer and a Loop B (LB) primer, wherein the FIP and BIP are present in a non-equal ratio such that molar concentration of BIP is highly skewed relative to molar concentration of FIP, wherein when FIP is present in a greater molar concentration than BIP, LF is present in a greater molar concentration than LB, and wherein when BIP is present in a greater molar concentration than FIP, LB is present in a greater molar concentration than LF; incubating the reaction mixture under amplification reaction conditions to produce a reaction product comprising amplified target nucleic acids, wherein the reaction product comprises a forward strand and a complementary backward strand, the forward strand comprising the FIP, the backward strand comprising the BIP; and detecting the amplified target nucleic acids by detection of specific binding of a fluorescence resonance energy transfer (FRET)-labeled oligonucleotide probe, wherein the oligonucleotide probe is a sequence-specific binding partner for a sequence present in the forward strand or the backward strand of the amplified target nucleic acids, wherein the oligonucleotide probe is a sequence-specific binding partner for a specified sequence present in the forward strand of the amplified target nucleic acids when FIP has a molar concentration at least 12% higher than the molar concentration of BIP, wherein the oligonucleotide probe is a sequence-specific binding partner for a specified sequence present in the backward strand of the amplified target nucleic acids when BIP has a molar concentration at least 12% higher than the molar concentration of FIP, wherein the FRET label comprises a quencher and a fluorescent moiety such that the quencher quenches a fluorescent signal of the fluorescent moiety when the oligonucleotide probe is in stem-loop configuration and not hybridized to the specified sequence, and wherein the fluorescent signal of the fluorescent moiety is detectable when the fluorescently-labeled oligonucleotide probe is specifically hybridized to the specified sequence.

Item 29. The method of item 28, wherein BIP has a molar concentration at least 50% higher than the molar concentration of FIP and no more than 300% higher than the molar concentration of FIP, or FIP has a molar concentration at least 50% higher than the molar concentration of BIP and no more than 300% higher than the molar concentration of BIP.

Item 30. The method of item 28 or 29, wherein the F3 and B3 are present in an equal ratio.

Item 31. The method of item 28 or 29, wherein the F3 and B3 are present in a non-equal ratio such that when FIP is present in a greater molar concentration than BIP, F3 is present in a greater molar concentration than B3, and wherein when BIP is present in a greater molar concentration than FIP, B3 is present in a greater molar concentration than F3.

Item 32. The method of item 31, wherein when BIP has a molar concentration at least 12% higher than the molar concentration of FIP and no more than 300% higher than the molar concentration of FIP, B3 has a molar concentration higher than the molar concentration of F3 and no more than 1000% higher than the molar concentration of F3.

Item 33. The method of item 31, wherein when BIP has a molar concentration at least 50% higher than the molar concentration of FIP and no more than 300% higher than the molar concentration of FIP, B3 has a molar concentration higher than the molar concentration of F3 and no more than 1000% higher than the molar concentration of F3.

Item 34. The method of item 31, wherein when FIP has a molar concentration at least 12% higher than the molar concentration of BIP and no more than 300% higher than the molar concentration of BIP, F3 has a molar concentration higher than the molar concentration of B3 and no more than 1000% higher than the molar concentration of B3.

Item 35. The method of item 31, wherein when FIP has a molar concentration at least 50% higher than the molar concentration of BIP and no more than 300% higher than the molar concentration of BIP, F3 has a molar concentration higher than the molar concentration of B3 and no more than 1000% higher than the molar concentration of B3.

Item 36. The method of item 28, wherein when BIP has a molar concentration at least 12% higher than the molar concentration of FIP and no more than 300% higher than the molar concentration of FIP, LB is present and no LF is present, or LB has a molar concentration at least 12% higher than the molar concentration of LF and no more than 500% higher than the molar concentration of LF when both LB and LF are present.

Item 37. The method of item 28, wherein when BIP has a molar concentration at least 50% higher than the molar concentration of FIP and no more than 300% higher than the molar concentration of FIP, LB is present and no LF is present, or LB has a molar concentration at least 50% higher than the molar concentration of LF and no more than 500% higher than the molar concentration of LF when both LB and LF are present.

Item 38. The method of item 28, wherein when FIP has a molar concentration at least 12% higher than the molar concentration of BIP and no more than 300% higher than the molar concentration of BIP, LF is present and no LB is present, or LF has a molar concentration at least 12% higher than the molar concentration of LB and no more than 500% higher than the molar concentration of LB when both LB and LF are present.

Item 39. The method of item 28, wherein when FIP has a molar concentration at least 50% higher than the molar concentration of BIP and no more than 300% higher than the molar concentration of BIP, LF is present and no LB is present, or LF has a molar concentration at least 50% higher than the molar concentration of LB and no more than 500% higher than the molar concentration of LB when both LB and LF are present.

Item 40. The method of any one of items 28 to 39, wherein the reaction mixture further comprises a reverse transcriptase.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method for detection of a target nucleic acid in a sample, comprising:
providing a reaction mixture comprising a LAMP assay primer set specific for the target nucleic acid, magnesium, dNTPs, a reaction buffer, a DNA polymerase, and a sample to be tested for presence of the target nucleic acid, wherein the LAMP assay primer set comprises a forward inner primer (FIP), a backward inner primer (BIP), a forward outer primer (F3) and a backward outer primer (B3), wherein the FIP and BIP are present in a non-equal ratio such that molar concentration of BIP is highly skewed relative to molar concentration of FIP;
incubating the reaction mixture under amplification reaction conditions to produce a reaction product comprising amplified target nucleic acids, wherein the reaction product comprises a forward strand and a complementary backward strand, the forward strand comprising the FIP, the backward strand comprising the BIP; and
detecting the amplified target nucleic acids.

2. The method of claim 1, wherein BIP has a molar concentration at least 12% higher than the molar concentration of FIP and no more than 300% higher than the molar concentration of FIP, or FIP has a molar concentration at least 12% higher than the molar concentration of BIP and no more than 300% higher than the molar concentration of BIP.

3. The method of claim 1, wherein BIP has a molar concentration at least 50% higher than the molar concentration of FIP and no more than 300% higher than the molar concentration of FIP, or FIP has a molar concentration at least 50% higher than the molar concentration of BIP and no more than 300% higher than the molar concentration of BIP.

4. The method of claim 1, wherein the F3 and B3 are present in an equal ratio.

5. The method of claim 1, wherein the F3 and B3 are present in a non-equal ratio such that when FIP is present in a greater molar concentration than BIP, F3 is present in a greater molar concentration than B3, and wherein when BIP is present in a greater molar concentration than FIP, B3 is present in a greater molar concentration than F3.

6. The method of claim 5, wherein when BIP has a molar concentration at least 12% higher than the molar concentration of FIP and no more than 300% higher than the molar concentration of FIP, B3 has a molar concentration higher than the molar concentration of F3 and no more than 1000% higher than the molar concentration of F3.

7. The method of claim 5, wherein when BIP has a molar concentration at least 50% higher than the molar concentration of FIP and no more than 300% higher than the molar concentration of FIP, B3 has a molar concentration higher than the molar concentration of F3 and no more than 1000% higher than the molar concentration of F3.

8. The method of claim 5, wherein when FIP has a molar concentration at least 12% higher than the molar concentration of BIP and no more than 300% higher than the molar concentration of BIP, F3 has a molar concentration higher than the molar concentration of B3 and no more than 1000% higher than the molar concentration of B3.

9. The method of claim 5, wherein when FIP has a molar concentration at least 50% higher than the molar concentration of BIP and no more than 300% higher than the molar concentration of BIP, F3 has a molar concentration higher than the molar concentration of B3 and no more than 1000% higher than the molar concentration of B3.

10. The method of claim 1, wherein the reaction mixture further comprises a Loop F (LF) primer, a Loop B (LB) primer, or both a Loop F (LF) primer and a Loop B (LB) primer, wherein the LF and LB are present in a non-equal ratio when both are present, such that molar concentration of LF is highly skewed relative to LB.

11.-14. (canceled)

15. The method of claim 1, wherein detecting the amplified target nucleic acids, comprises detecting one or more of: pH, turbidity, an electrophoresis pattern, and a detectable label.

16. The method of claim 15, wherein detecting the amplified target nucleic acids comprises detecting a detectable label, and wherein the detectable label comprises one or more of: a fluorescent label, a bioluminescent label, a chemiluminescent label, a chromophore, a magnetic label, an enzyme, a substrate, and a radioisotope.

17. The method of claim 15, wherein the detectable label comprises a fluorescence resonance energy transfer (FRET) fluorescent label.

18. The method of claim 15, wherein the detectable label comprises a fluorescent label comprising a fluorescent intercalating dye.

19. The method of claim 15, wherein the detectable label is present in one or more primers of the LAMP assay primer set.

20. The method of claim 16, wherein the detectable label is a fluorescent label present in one or more primers of the LAMP assay primer set, incorporated into the amplified target nucleic acids.

21. The method of claim 15, wherein the detectable label is a fluorescent label present in a probe, and wherein the probe is a sequence-specific binding partner for a specified sequence present in the amplified target nucleic acids.

22. The method of claim 21, wherein the probe is a sequence-specific binding partner for a specified sequence present in the forward strand of the amplified target nucleic acids when FIP has a molar concentration at least 12% higher than the molar concentration of BIP and no more than 300% higher than the molar concentration of BIP.

23.-25. (canceled)

26. The method of claim 21, wherein the probe is a fluorescence resonance energy transfer (FRET)-labeled oligonucleotide probe, wherein the FRET label comprises a quencher and a fluorescent moiety such that the quencher quenches a fluorescent signal of the fluorescent moiety when the oligonucleotide probe is in stem-loop configuration and not hybridized to the specified sequence, and wherein the fluorescent signal of the fluorescent moiety is detectable when the fluorescently-labeled oligonucleotide probe is specifically hybridized to the specified sequence.

27. The method of claim 1, wherein the reaction mixture further comprises a reverse transcriptase.

28. A method for detection of a target nucleic acid in a sample, comprising:
providing a reaction mixture comprising: a LAMP assay primer set specific for the target nucleic acid, magnesium, dNTPs, a reaction buffer, a DNA polymerase, and a sample to be tested for presence of the target nucleic acid, wherein the LAMP assay primer set comprises 1) a forward inner primer (FIP), 2) a backward inner primer (BIP), 3) a forward outer primer (F3), 4) a backward outer primer (B3), and 5) a Loop F (LF) primer or a Loop B (LB) primer, or both a Loop F (LF) primer and a Loop B (LB) primer, wherein the FIP and BIP are present in a non-equal ratio such that molar concentration of BIP is highly skewed relative to molar concentration of FIP, wherein when FIP is present in a greater molar concentration than BIP, LF is present in a greater molar concentration than LB, and wherein when BIP is present in a greater molar concentration than FIP, LB is present in a greater molar concentration than LF;
incubating the reaction mixture under amplification reaction conditions to produce a reaction product comprising amplified target nucleic acids, wherein the reaction product comprises a forward strand and a complementary backward strand, the forward strand comprising the FIP, the backward strand comprising the BIP; and
detecting the amplified target nucleic acids by detection of specific binding of a fluorescence resonance energy transfer (FRET)-labeled oligonucleotide probe, wherein the oligonucleotide probe is a sequence-specific binding partner for a sequence present in the forward strand or the backward strand of the amplified target nucleic acids, wherein the oligonucleotide probe is a sequence-specific binding partner for a specified sequence present in the forward strand of the amplified target nucleic acids when FIP has a molar concentration at least 12% higher than the molar concentration of BIP, wherein the oligonucleotide probe is a sequence-specific binding partner for a specified sequence present in the backward strand of the amplified target nucleic acids when BIP has a molar concentration at least 12% higher than the molar concentration of FIP, wherein the FRET label comprises a quencher and a fluorescent moiety such that the quencher quenches a fluorescent signal of the fluorescent moiety when the oligonucleotide probe is in stem-loop configuration and not hybridized to the specified sequence, and wherein the fluorescent signal of the fluorescent moiety is detectable when the fluorescently-labeled oligonucleotide probe is specifically hybridized to the specified sequence.

29.-40. (canceled)
